(19) United States
(12) Patent Application Publication
(10) Pub. No.: US 2026/0015323 A1
(43) Pub. Date: Jan. 15, 2026

(54) PIPERIDINE COMPOUNDS

(71) Applicant: Telo Therapeutics, Inc., San Francisco, CA (US)

(72) Inventors: Katherine Widdowson, San Diego, CA (US); Robert Joseph Allen Bell, San Francisco, CA (US)

(21) Appl. No.: 19/266,910

(22) Filed: Jul. 11, 2025

Related U.S. Application Data
(60) Provisional application No. 63/671,343, filed on Jul. 15, 2024.

(51) Int. Cl. C07D 211/34; A61K 31/445; A61K 31/451

(52) U.S. Cl. CPC C07D 211/34; A61K 31/445; A61K 31/451

(57) ABSTRACT

The present invention relates to piperidine compounds, and pharmaceutical compositions of the same, that are inhibitors of XPO1 and are useful in the treatment of diseases such as cancer.

### CROSS REFERENCE TO RELATED APPLICATION

This application claims priority to U.S. Provisional Application No. 63/671,343, filed Jul. 15, 2024, the entire content of which application is hereby incorporated by reference in its entirety.

### FIELD OF THE INVENTION

The present invention relates to piperidine compounds, and pharmaceutical compositions of the same, that are inhibitors of XPO1 and are useful in the treatment of diseases such as cancer.

### BACKGROUND OF THE INVENTION

Exportin 1 (XPO1), also known as chromosomal region maintenance 1 (CRM1), is a nuclear export receptor that mediates the transport of many proteins and RNA species from the cell nucleus to the cytoplasm. The hundreds of proteins XPO1 is involved in transporting include tumor suppressors, oncoproteins, and regulators of cell growth. XPO1 is often overexpressed in many types of cancers, and its function in shuttling cargo misregulated. As such, XPO1 has been identified as a promising cancer therapeutic target.

Compounds that inhibit XPO1 can block the transport of proteins involved in cancer cell growth, leading to inhibition of cell proliferation and eventual apoptosis of cancer cells. One compound that selectively inhibits XPO1, Selinexor (KPT-330), has received FDA approval for resistant and relapsed multiple myeloma (in combination with bortezomib and dexamethasone) and relapsed/refractory diffuse large B-cell lymphoma (DLBCL), and has shown potential in clinical trials for the treatment of other cancers. Selinexor covalently binds to Cys528 in the cargo-binding groove of XPO1 in order to prevent cargo binding and export. However, the presence of systemic toxicities has limited the clinical application of Selinexor.

In addition to proteins related to cancer cell growth, XPO1 is involved in the transport of many other important proteins, including those involved in inflammatory and viral pathways. XPO1 also mediates the transport of many species of RNA, and may have a large impact on different aspects of RNA metabolism.

Inhibition of XPO1-mediated nuclear transport has been shown to be a promising therapeutic strategy for treating cancers and other conditions. This application is directed to inhibitors of XPO1.

### SUMMARY OF THE INVENTION

Provided herein are piperidine compounds that are inhibitors of XPO1. In particular, provided herein are compounds of Formula (I).

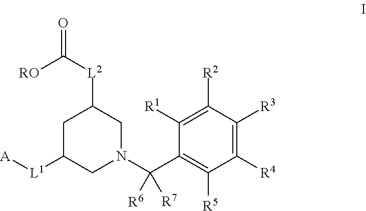

or pharmaceutically acceptable salts thereof, wherein the variables are as defined herein.

The present disclosure further provides pharmaceutical compositions comprising a compound of Formula I, or a pharmaceutically acceptable salt thereof, and at least one pharmaceutically acceptable excipient.

The present disclosure further provides methods of inhibiting an activity of XPO1 comprising contacting the XPO1 with a compound of Formula I, or a pharmaceutically acceptable salt thereof.

The present disclosure further provides a method of treating or preventing a disease in a patient wherein the disease is characterized by overexpression or upregulation of XPO1, comprising administering to the patient a therapeutically effective amount of a compound of Formula I, or a pharmaceutically acceptable salt thereof.

### BRIEF DESCRIPTION OF THE FIGURES

FIG. 1A is a western blot indicating the expression of XPO1 and HSP70 protein levels in the presence of Compound 21, stereisomer 2; Compound 31, stereisomer 1; Compound 16, stereoisomer 2; and DMSO control.

FIG. 1B is a western blot indicating the expression of XPO1 and HSP70 protein levels in the presence of Compound 16, enantiomer 1-A; Compound 21, stereoisomer 1; and DMSO control.

FIG. 1C is a western blot indicating the expression of XPO1 and HSP70 protein levels in the presence of Compound 25, second-eluting isomer, and DMSO control.

### DETAILED DESCRIPTION

### Compounds

The present disclosure provides compounds of Formula I:

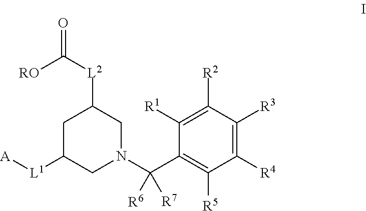

or a pharmaceutically acceptable salt thereof, wherein:
A is C₃₋₁₀ alkyl, C₃₋₁₀ cycloalkyl, 3-14 membered heterocycloalkyl, or phenyl, each optionally substituted with 1, 2, or 3 R^{A};
L¹ is absent, C₁₋₈ alkylene, C₃₋₈ cycloalkylene, or C₂₋₈ alkenylene, wherein the C₁₋₈ alkylene, C₃₋₈ cycloalkylene, or C₂₋₈ alkenylene is optionally substituted by 1, 2, or 3 substituents independently selected from C₁₋₄ alkyl, C₁₋₄haloalkyl, C₁₋₄ alkoxy, F, Cl, Br, and OH;
L² is methylene, C₂₋₈ alkenylene, or C₃₋₈ cycloalkylene, each optionally substituted by 1, 2, or 3 substituents independently selected from C₁₋₄ alkyl, C₁₋₄ haloalkyl, C₁₋₄ alkoxy, F, Cl, Br, and OH;
R is H, C₁₋₈ alkyl, or aryl-C₁₋₄ alkyl, wherein the C₁₋₈ alkyl and aryl-C₁₋₄ alkyl are optionally substituted by 1, 2, or 3 substituents independently selected from C₁₋₄ alkyl, C₁₋₄ haloalkyl, halo, CN, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
R¹, R², R³, R⁴, and R⁵ are each independently selected from H, C₁₋₄ alkyl, C₁₋₄ haloalkyl, C₂₋₆ alkenyl, C₂₋₆ alkynyl, C₃₋₄ cycloalkyl, 3-4 membered heterocycloalkyl, halo, CN, NO₂, N₃, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein the C₁₋₄ alkyl group of R¹, R², R³, R⁴, and R⁵ is optionally substituted by 1, 2, or 3 substituents independently selected from C₃₋₄ cycloalkyl, 3-4 membered heterocycloalkyl, halo, CN, NO₂, N₃, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR_{c1}R^{d1}, S(O)R^{b1}, S(O)NR^{c1}R^{d1}, S(O)₂R^{b1}, and S(O)₂NR^{c1}R^{d1};
or R¹ and R² together with the carbon atoms to which they are attached form a fused C₃₋₇ cycloalkyl ring, a fused 3-7 membered heterocycloalkyl ring, a fused phenyl ring, or a fused 5-6 membered heteroaryl ring, each optionally substituted by 1, 2, or 3 substituents independently selected from C₁₋₄ alkyl, C₁₋₄haloalkyl, C₂₋₆ alkenyl, C₂₋₆ alkynyl, C₃₋₄ cycloalkyl, 3-4 membered heterocycloalkyl, halo, CN, NO₂, N₃, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
or R² and R³ together with the carbon atoms to which they are attached form a fused C₃₋₇ cycloalkyl ring, a fused 3-7 membered heterocycloalkyl ring, a fused phenyl ring, or a fused 5-6 membered heteroaryl ring, each optionally substituted by 1, 2, or 3 substituents independently selected from C₁₋₄ alkyl, C₁₋₄haloalkyl, C₂₋₆ alkenyl, C₂₋₆ alkynyl, C₃₋₄ cycloalkyl, 3-4 membered heterocycloalkyl, halo, CN, NO₂, N₃, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
R⁶ and R⁷ are each independently selected from H, methyl, ethyl, CF₃, and CN; each R^{A} is independently selected from halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2} S(O)R^{b2}, S(O)NR^{c2}R^{d2}, S(O)₂R^{b2}, and S(O)₂NR^{c2}R^{d2}, wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{A} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2} NR^{c2}S(O)₂NR^{c2}R^{d2} S(O)R^{b2}, S(O)NR^{c2}R^{d2}, S(O)₂R^{b2}, and S(O)₂NR^{c2}R^{d2};
each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3} NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3} NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each R^{a}, R^{b}, R^{c}, R^{d}, R^{a1}, R^{b1}, R^{c1}, R^{d1}, R^{a2}, R^{b2}, R^{c2}, R^{d2}, R^{a3}, R^{b3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a}, R^{b}, R^{c}, R^{d}, R^{a1}, R^{b1}, R^{c1}, R^{d1}, R^{a2}, R^{b2}, R^{c2}, R^{d2}, R^{a3}, R^{b3}, R^{c3}, and R^{d3} is is optionally substituted with 1, 2, 3, 4, or 5 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
or R^{c} and R^{d} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
or R^{c1} and R^{d1} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
or R^{c2} and R^{d2} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
or R^{c3} and R^{d3} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
each R^{a4}, R^{b4}, R^{c4}, and R^{d4} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy; and
each R^{e}, R^{e1}, R^{e2}, R^{e3}, and R^{e4} is independently selected from H, C₁₋₄ alkyl, and CN;
wherein when A is phenyl optionally substituted with 1, 2, or 3 R^{A}, and L¹ is absent or C₁₋₂ alkylene, then L² is other than methylene optionally substituted by 1, 2, or 3 substituents independently selected from F, Cl, Br, OH, and methoxy.

In some embodiments, A is C₃₋₁₀ alkyl optionally substituted with 1, 2, or 3 R^{A}. In some embodiments, A is C₃₋₇ alkyl optionally substituted with 1 or 2 R^{A}. In some embodiments, A is isopropyl, 2-methylprop-1-yl, tert-butyl, 3-methylbut-2-yl, or 2,4-dimethylpent-3-yl. In some embodiments, A is isopropyl.

In some embodiments, A is C₃₋₁₀ cycloalkyl optionally substituted with 1, 2, or 3 R^{A}. In some embodiments, A is C₃₋₈ cycloalkyl optionally substituted with 1 R^{A}. In some embodiments, A is cyclopropyl, cyclohexyl, cycloheptyl, or cyclooctyl, each optionally substituted with 1 R^{A}. In some embodiments, A is C₆₋₈ cycloalkyl. In some embodiments, A is cyclohexyl, cycloheptyl, or cyclooctyl. In some embodiments, A is cycloheptyl.

In some embodiments, A is 3-14 membered heterocycloalkyl optionally substituted with 1, 2, or 3 R^{A}. In some embodiments, A is 6 or 7-membered heterocycloalkyl optionally substituted with 1 R^{A}. In some embodiments, A is tetrahydropyranyl or 1,3-dioxepan-2-yl optionally substituted with 1 R^{A}. In some embodiments, A is 1,3-dioxepan-2-yl optionally substituted with 1 R^{A}.

In some embodiments, A is phenyl optionally substituted with 1, 2, or 3 R^{A}. In some embodiments, A is phenyl optionally substituted with 1 R^{A}.

In some embodiments, L¹ is absent.

In some embodiments, L¹ is methylene.

In some embodiments, L¹ is C₂₋₈ alkenylene, optionally substituted by 1 or 2 substituents independently selected from C₁₋₄ alkyl, C₁₋₄haloalkyl, C₁₋₄ alkoxy, F, Cl, Br, and OH. In some embodiments, L¹ is ethenylene.

In some embodiments, L² is methylene optionally substituted by 1 or 2 substituents independently selected from C₁₋₄ alkyl, C₁₋₄haloalkyl, C₁₋₄ alkoxy, F, Cl, Br, and OH.

In some embodiments, L² is C₂₋₈ alkenylene optionally substituted by 1, 2, or 3 substituents independently selected from C₁₋₄ alkyl, C₁₋₄ haloalkyl, C₁₋₄ alkoxy, F, Cl, Br, and OH.

In some embodiments, L² is C₃₋₈ cycloalkylene optionally substituted by 1, 2, or 3 substituents independently selected from C₁₋₄ alkyl, C₁₋₄ haloalkyl, C₁₋₄ alkoxy, F, Cl, Br, and OH.

In some embodiments, L² is methylene or C₃₋₈ cycloalkylene. In some embodiments, L² is methylene or cyclopropylene. In some embodiments, L² is methylene.

In some embodiments, R is H.

In some embodiments, R is C₁₋₈ alkyl or aryl-C₁₋₄ alkyl, wherein the C₁₋₈ alkyl and aryl-C₁₋₄ alkyl are optionally substituted by 1, 2, or 3 substituents independently selected from C₁₋₄ alkyl, C₁₋₄ haloalkyl, halo, CN, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e1})R^{b}, C(═NR^{e1})NR^{c}R^{d}, NR^{c}C(═NR^{e})NR^{c}R^{d} NR^{c}S(O)R^{b1}, NR^{c}S(O)₂R^{b}, NR^{c}S(O)₂NR^{c}R^{d}, S(O)R^{b1}, S(O)NR^{c}R^{d}, S(O)₂R^{b}, and S(O)₂NR^{c}R^{d}.

In some embodiments, R³ is selected from C₁₋₄ alkyl, C₁₋₄haloalkyl, C₂₋₆ alkenyl, C₂₋₆ alkynyl, C₃₋₄ cycloalkyl, 3-4 membered heterocycloalkyl, halo, CN, NO₂, N₃, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein the C₁₋₄ alkyl group of R¹, R², R³, R⁴, and R⁵ is optionally substituted by 1, 2, or 3 substituents independently selected from C₃₋₄ cycloalkyl, 3-4 membered heterocycloalkyl, halo, CN, NO₂, N₃, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR_{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

In some embodiments, R³ is C₁₋₄haloalkyl. In some embodiments, R³ is trifluoromethyl.

In some embodiments, R¹, R², R⁴, and R⁵ are each H.

In some embodiments, R⁶ and R⁷ are each H.

In some embodiments, each R^{A} is independently selected from C₁₋₆ alkyl, C₁₋₆ haloalkyl, and OR^{a2}.

In some embodiments, each R^{a2} is selected from H and C₁₋₆ alkyl.

In some embodiments, when A is phenyl optionally substituted with 1, 2, or 3 R^{A}, and L¹ is absent or C₁₋₂ alkylene, then L² is other than methylene or C₂₋₈ alkylene, each optionally substituted by 1, 2, or 3 substituents independently selected from F, Cl, Br, OH, and methoxy.

In some embodiments:
L¹ is absent, C₁₋₈ alkylene, or C₂₋₈ alkenylene, wherein the C₁₋₈ alkylene or C₂₋₈ alkenylene is optionally substituted by 1, 2, or 3 substituents independently selected from C₁₋₄ alkyl, C₁₋₄haloalkyl, C₁₋₄ alkoxy, F, Cl, Br, and OH;
L² is methylene or C₃₋₈ cycloalkylene, each optionally substituted by 1, 2, or 3 substituents independently selected from C₁₋₄ alkyl, C₁₋₄ haloalkyl, C₁₋₄ alkoxy, F, Cl, Br, and OH;
R is H;
R¹, R², R⁴, and R⁵ are each H;
R³ is selected from C₁₋₄ alkyl, C₁₋₄ haloalkyl, C₂₋₆ alkenyl, C₂₋₆ alkynyl, C₃₋₄ cycloalkyl, 3-4 membered heterocycloalkyl, halo, CN, NO₂, N₃, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR_{c1}R^{d1}, S(O)R^{b1}, S(O)NR^{c1}R^{d1}, S(O)₂R^{b1}, and S(O)₂NR^{c1}R^{d1}; wherein the C₁₋₄ alkyl group of R¹, R², R³, R⁴, and R⁵ is optionally substituted by 1, 2, or 3 substituents independently selected from C₃₋₄ cycloalkyl, 3-4 membered heterocycloalkyl, halo, CN, NO₂, N₃, OR^{a1}, SR^{a1}, C(O)R^{b1}, C(O)NR^{c1}R^{d1}, C(O)OR^{a1}, OC(O)R^{b1}, OC(O)NR_{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═R^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; and
   R⁶ and R⁷ are each H.

In some embodiments:
L¹ is absent, C₁₋₈ alkylene, or C₂₋₈ alkenylene, wherein the C₁₋₈ alkylene or C₂₋₈ alkenylene is optionally substituted by 1 or 2 substituents independently selected from C₁₋₄ alkyl, C₁₋₄haloalkyl, C₁₋₄ alkoxy, F, Cl, Br, and OH;
L² is methylene or C₃₋₈ cycloalkylene;
R is H;
R¹, R², R³, R⁴, and R⁵ are each independently selected from H and C₁₋₄ haloalkyl; and
R⁶ and R⁷ are each H.

In some embodiments:
A is C₃₋₁₀ alkyl, C₃₋₁₀ cycloalkyl, 3-14 membered heterocycloalkyl, or phenyl, each optionally substituted with 1 R^{A};
L¹ is absent, methylene, or C₂₋₈ alkenylene, wherein the methylene or C₂₋₈ alkenylene is optionally substituted by 1 or 2 substituents independently selected from C₁₋₄ alkyl, C₁₋₄ alkoxy, and OH;
L² is methylene or C₃₋₈ cycloalkylene;
R is H;
R¹, R², R⁴, and R⁵ are each H;
R³ is C₁₋₄ haloalkyl;
R⁶ and R⁷ are each H;
each R^{A} is independently selected from C₁₋₆ alkyl, C₁₋₆ haloalkyl, and OR^{a2}; and
each R^{a2} is selected from H and C₁₋₆ alkyl.

In some embodiments, the compound of Formula I is a compound of Formula II:

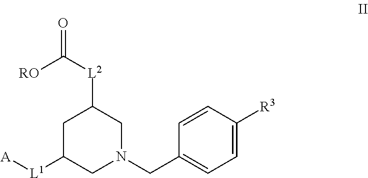

or a pharmaceutically acceptable salt thereof, wherein variables A, L¹, L², and R³ are defined according to the definitions provided herein for compounds of Formula I.

In some embodiments, the compound of Formula I is a compound of Formula III:

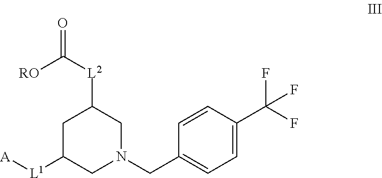

or a pharmaceutically acceptable salt thereof, wherein variables A, L¹, and L² are defined according to the definitions provided herein for compounds of Formula I.

In some embodiments, the compound of Formula I is a compound of Formula IV:

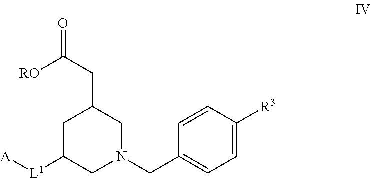

or a pharmaceutically acceptable salt thereof, wherein variables A, L¹, and R³ are defined according to the definitions provided herein for compounds of Formula I.

In some embodiments, the compound of Formula I is a compound of Formula V:

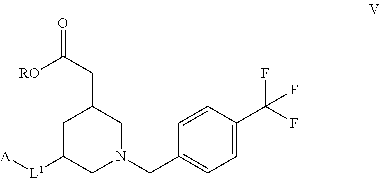

or a pharmaceutically acceptable salt thereof, wherein variables A and L¹ are defined according to the definitions provided herein for compounds of Formula I.

In some embodiments, the compound of Formula I is a compound of Formula VI:

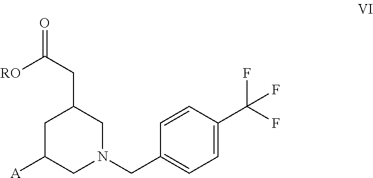

or a pharmaceutically acceptable salt thereof, wherein variable A is defined according to the definitions provided herein for compounds of Formula I. In some embodiments, A is C₃₋₁₀ cycloalkyl.

In some embodiments, the compound provided herein (e.g., a compound of Formula I) is selected from:
2-(5-cyclooctyl-1-(4-(trifluoromethyl)benzyl)piperidin-3-yl)acetic acid;
2-(1-(4-(trifluoromethyl)benzyl)-5-(4-(trifluoromethyl)phenyl)piperidin-3-yl)cyclopropane-1-carboxylic acid;
2-(5-cycloheptyl-1-(4-(trifluoromethyl)benzyl)piperidin-3-yl)acetic acid;
2-(1-(4-(trifluoromethyl)benzyl)-5-(4-(trifluoromethyl)styryl)piperidin-3-yl)acetic acid;
2-(5-cyclohexyl-1-(4-(trifluoromethyl)benzyl)piperidin-3-yl)acetic acid; and
2-(5-isopropyl-1-(4-(trifluoromethyl)benzyl)piperidin-3-yl)acetic acid;
   or a pharmaceutically acceptable salt thereof.

In some embodiments, the compound provided herein (e.g., a compound of Formula I) is selected from:
2-((3R,5S)-5-cyclooctyl-1-(4-(trifluoromethyl)benzyl)piperidin-3-yl)acetic acid;
2-((3S,5R)-5-cyclooctyl-1-(4-(trifluoromethyl)benzyl)piperidin-3-yl)acetic acid;
2-((3S,5S)-5-cyclooctyl-1-(4-(trifluoromethyl)benzyl)piperidin-3-yl)acetic acid;
2-((3R,5R)-5-cyclooctyl-1-(4-(trifluoromethyl)benzyl)piperidin-3-yl)acetic acid;
(2R)-2-((3S,5S)-1-(4-(trifluoromethyl)benzyl)-5-(4-(trifluoromethyl)phenyl)piperidin-3-yl)cyclopropane-1-carboxylic acid;
(2R)-2-((3R,5R)-1-(4-(trifluoromethyl)benzyl)-5-(4-(trifluoromethyl)phenyl)piperidin-3-yl)cyclopropane-1-carboxylic acid;
(2R)-2-((3S,5R)-1-(4-(trifluoromethyl)benzyl)-5-(4-(trifluoromethyl)phenyl)piperidin-3-yl)cyclopropane-1-carboxylic acid;
(2R)-2-((3R,5S)-1-(4-(trifluoromethyl)benzyl)-5-(4-(trifluoromethyl)phenyl)piperidin-3-yl)cyclopropane-1-carboxylic acid;
(2S)-2-((3S,5S)-1-(4-(trifluoromethyl)benzyl)-5-(4-(trifluoromethyl)phenyl)piperidin-3-yl)cyclopropane-1-carboxylic acid;
(2S)-2-((3R,5R)-1-(4-(trifluoromethyl)benzyl)-5-(4-(trifluoromethyl)phenyl)piperidin-3-yl)cyclopropane-1-carboxylic acid;
(2S)-2-((3S,5R)-1-(4-(trifluoromethyl)benzyl)-5-(4-(trifluoromethyl)phenyl)piperidin-3-yl)cyclopropane-1-carboxylic acid;
(2S)-2-((3R,5S)-1-(4-(trifluoromethyl)benzyl)-5-(4-(trifluoromethyl)phenyl)piperidin-3-yl)cyclopropane-1-carboxylic acid;
2-((3S,5S)-1-(4-(trifluoromethyl)benzyl)-5-((E)-4-(trifluoromethyl)styryl)piperidin-3-yl)acetic acid;
2-((3R,5R)-1-(4-(trifluoromethyl)benzyl)-5-((E)-4-(trifluoromethyl)styryl)piperidin-3-yl)acetic acid;
2-((3S,5R)-1-(4-(trifluoromethyl)benzyl)-5-((E)-4-(trifluoromethyl)styryl)piperidin-3-yl)acetic acid;
2-((3R,5S)-1-(4-(trifluoromethyl)benzyl)-5-((E)-4-(trifluoromethyl)styryl)piperidin-3-yl)acetic acid;
2-((3R,5S)-5-cycloheptyl-1-(4-(trifluoromethyl)benzyl)piperidin-3-yl)acetic acid;
2-((3S,5R)-5-cycloheptyl-1-(4-(trifluoromethyl)benzyl)piperidin-3-yl)acetic acid;
2-((3R,5R)-5-cycloheptyl-1-(4-(trifluoromethyl)benzyl)piperidin-3-yl)acetic acid;
2-((3S,5S)-5-cycloheptyl-1-(4-(trifluoromethyl)benzyl)piperidin-3-yl)acetic acid;
2-((3R,5S)-5-cyclohexyl-1-(4-(trifluoromethyl)benzyl)piperidin-3-yl)acetic acid;
2-((3S,5R)-5-cyclohexyl-1-(4-(trifluoromethyl)benzyl)piperidin-3-yl)acetic acid;
2-((3R,5R)-5-cyclohexyl-1-(4-(trifluoromethyl)benzyl)piperidin-3-yl)acetic acid;
2-((3S,5S)-5-cyclohexyl-1-(4-(trifluoromethyl)benzyl)piperidin-3-yl)acetic acid;
2-((3R,5S)-5-isopropyl-1-(4-(trifluoromethyl)benzyl)piperidin-3-yl)acetic acid;
2-((3S,5R)-5-isopropyl-1-(4-(trifluoromethyl)benzyl)piperidin-3-yl)acetic acid;
2-((3R,5R)-5-isopropyl-1-(4-(trifluoromethyl)benzyl)piperidin-3-yl)acetic acid; and
2-((3S,5S)-5-isopropyl-1-(4-(trifluoromethyl)benzyl)piperidin-3-yl)acetic acid;
   or a pharmaceuticlly acceptable salt thereof.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “pyridinyl,” “pyridyl,” or “a pyridine ring” may refer to a pyridin-2-yl, pyridin-3-yl, or pyridin-4-yl ring.

The term “n-membered,” where “n” is an integer, typically describes the number of ring-forming atoms in a moiety where the number of ring-forming atoms is “n”. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

For compounds of the invention in which a variable appears more than once, each variable can be a different moiety independently selected from the group defining the variable. For example, where a structure is described having two R groups that are simultaneously present on the same compound, the two R groups can represent different moieties independently selected from the group defined for R.

As used herein, the phrase “optionally substituted” means unsubstituted or substituted.

As used herein, the term “substituted” means that a hydrogen atom is replaced by a non-hydrogen group. It is to be understood that substitution at a given atom is limited by valency.

As used herein, the term “Cᵢ₋ⱼ,” where i and j are integers, employed in combination with a chemical group, designates a range of the number of carbon atoms in the chemical group with i-j defining the range. For example, C₁₋₆ alkyl refers to an alkyl group having 1, 2, 3, 4, 5, or 6 carbon atoms.

As used herein, the term “alkyl,” employed alone or in combination with other terms, refers to a saturated hydrocarbon group that may be straight-chain or branched. In some embodiments, the alkyl group contains 1 to 7, 1 to 6, 1 to 4, or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, 2,4-dimethyl-3-pentyl, n-hexyl, 1,2,2-trimethylpropyl, n-heptyl, and the like. In some embodiments, the alkyl group is methyl, ethyl, or propyl.

As used herein, the term “alkylene,” employed alone or in combination with other terms, refers to a linking alkyl group.

As used herein, “alkenyl,” employed alone or in combination with other terms, refers to an alkyl group having one or more carbon-carbon double bonds. In some embodiments, the alkenyl moiety contains 2 to 6 or 2 to 4 carbon atoms. Example alkenyl groups include, but are not limited to, ethenyl, n-propenyl, isopropenyl, n-butenyl, sec-butenyl, and the like.

As used herein, the term “alkenylene,” employed alone or in combination with other terms, refers to a linking alkenyl group.

As used herein, “alkynyl,” employed alone or in combination with other terms, refers to an alkyl group having one or more carbon-carbon triple bonds. Example alkynyl groups include, but are not limited to, ethynyl, propyn-1-yl, propyn-2-yl, and the like. In some embodiments, the alkynyl moiety contains 2 to 6 or 2 to 4 carbon atoms.

As used herein, “halo” or “halogen”, employed alone or in combination with other terms, includes fluoro, chloro, bromo, and iodo. In some embodiments, halo is F or Cl.

As used herein, the term “haloalkyl,” employed alone or in combination with other terms, refers to an alkyl group having up to the full valency of halogen atom substituents, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F₅, CHF₂, CCl₃, CHCl₂, C₂Cl₅, and the like.

As used herein, the term “alkoxy,” employed alone or in combination with other terms, refers to a group of formula —O-alkyl. Example alkoxy groups include methoxy, ethoxy, propoxy (e.g., n-propoxy and isopropoxy), t-butoxy, and the like. In some embodiments, the alkyl group has 1 to 6 or 1 to 4 carbon atoms.

As used herein, “haloalkoxy,” employed alone or in combination with other terms, refers to a group of formula —O-(haloalkyl). In some embodiments, the haloalkoxy group has 1 to 6 or 1 to 4 carbon atoms. An example haloalkoxy group is —OCF₃.

As used herein, “amino,” employed alone or in combination with other terms, refers to NH₂.

As used herein, the term “cycloalkyl,” employed alone or in combination with other terms, refers to a non-aromatic cyclic hydrocarbon including cyclized alkyl and alkenyl groups. Cycloalkyl groups can include mono- or polycyclic (e.g., having 2, 3, or 4 fused, bridged, or spiro rings) ring systems. Also included in the definition of cycloalkyl are moieties that have one or more aromatic rings (e.g., aryl or heteroaryl rings) fused (i.e., having a bond in common with) to the cycloalkyl ring, for example, benzo derivatives of cyclopentane, cyclohexene, cyclohexane, and the like, or pyrido derivatives of cyclopentane or cyclohexane. Ring-forming carbon atoms of a cycloalkyl group can be optionally substituted by oxo. Cycloalkyl groups also include cycloalkylidenes. The term “cycloalkyl” also includes bridgehead cycloalkyl groups (e.g., non-aromatic cyclic hydrocarbon moieties containing at least one bridgehead carbon, such as admantan-1-yl) and spirocycloalkyl groups (e.g., non-aromatic hydrocarbon moieties containing at least two rings fused at a single carbon atom, such as spiro[2.5]octane and the like). In some embodiments, the cycloalkyl group has 3 to 10 ring members, or 3 to 7 ring members. In some embodiments, the cycloalkyl group is monocyclic or bicyclic. In some embodiments, the cycloalkyl group is monocyclic. In some embodiments, the cycloalkyl group is a C₃₋₇ monocyclic cycloalkyl group. Example cycloalkyl groups include cyclopropyl, cyclobutyl, cyclopentyl, cyclohexyl, cycloheptyl, cyclooctyl, cyclopentenyl, cyclohexenyl, cyclohexadienyl, cycloheptatrienyl, norbornyl, norpinyl, norcarnyl, tetrahydronaphthalenyl, octahydronaphthalenyl, indanyl, and the like. In some embodiments, the cycloalkyl group is cyclopropyl, cyclobutyl, cyclopentyl, or cyclohexyl. In some embodiments, the cycloalkyl group is cyclopropyl, cyclohexyl, cycloheptyl, or cyclooctyl.

As used herein, the term “cycloalkylene,” employed alone or in combination with other terms, refers to a linking cycloalkyl group.

As used herein, the term “heterocycloalkyl,” employed alone or in combination with other terms, refers to a non-aromatic ring or ring system, which may optionally contain one or more alkenylene or alkynylene groups as part of the ring structure, which has at least one heteroatom ring member independently selected from nitrogen, sulfur, oxygen, and phosphorus.

Heterocycloalkyl groups can include mono- or polycyclic (e.g., having 2, 3 or 4 fused, bridged, or spiro rings) ring systems. In some embodiments, the heterocycloalkyl group is a monocyclic or bicyclic group having 1, 2, 3, or 4 heteroatoms independently selected from nitrogen, sulfur and oxygen. Also included in the definition of heterocycloalkyl are moieties that have one or more aromatic rings (e.g., aryl or heteroaryl rings) fused (i.e., having a bond in common with) to the non-aromatic heterocycloalkyl ring, for example, 1,2,3,4-tetrahydro-quinoline and the like. Where the heterocycloalkyl group includes a fused aromatic ring, the heterocycloalkyl group can be attached to the main structure though either the aromatic or non-aromatic ring. Heterocycloalkyl groups can also include bridgehead heterocycloalkyl groups (e.g., a heterocycloalkyl moiety containing at least one bridgehead atom, such as azaadmantan-1-yl and the like) and spiroheterocycloalkyl groups (e.g., a heterocycloalkyl moiety containing at least two rings fused at a single atom, such as [1,4-dioxa-8-aza-spiro[4.5]decan-N-yl] and the like). In some embodiments, the heterocycloalkyl group has 3 to 10 ring-forming atoms, 4 to 10 ring-forming atoms, or about 3 to 8 ring forming atoms. In some embodiments, the heterocycloalkyl group has 2 to 20 carbon atoms, 2 to 15 carbon atoms, 2 to 10 carbon atoms, or about 2 to 8 carbon atoms. In some embodiments, the heterocycloalkyl group has 1 to 5 heteroatoms, 1 to 4 heteroatoms, 1 to 3 heteroatoms, or 1 to 2 heteroatoms. The carbon atoms or heteroatoms in the ring(s) of the heterocycloalkyl group can be oxidized to form a carbonyl, an N-oxide, or a sulfonyl group (or other oxidized linkage) or a nitrogen atom can be quaternized. In some embodiments, the heterocycloalkyl portion is a C₂₋₇ monocyclic heterocycloalkyl group. In some embodiments, the heterocycloalkyl group is a morpholine ring, pyrrolidine ring, piperazine ring, piperidine ring, tetrahydropyran ring, tetrahyropyridine, azetidine ring, tetrahydrofuran ring, or a dioxepanyl ring.

As used herein, the term “aryl,” employed alone or in combination with other terms, refers to a monocyclic or polycyclic (e.g., a fused ring system) aromatic hydrocarbon moiety, such as, but not limited to, phenyl, 1-naphthyl, 2-naphthyl, and the like. In some embodiments, aryl groups have from 6 to 10 carbon atoms or 6 carbon atoms. In some embodiments, the aryl group is a monocyclic or bicyclic group. In some embodiments, the aryl group is phenyl or naphthyl.

As used herein, the term “heteroaryl,” employed alone or in combination with other terms, refers to a monocyclic or polycyclic (e.g., a fused ring system) aromatic ring moiety, having one or more heteroatom ring members independently selected from nitrogen, sulfur and oxygen. In some embodiments, the heteroaryl group is a monocyclic or a bicyclic group having 1, 2, 3, or 4 heteroatoms independently selected from nitrogen, sulfur and oxygen. Example heteroaryl groups include, but are not limited to, pyridyl, pyrimidinyl, pyrazinyl, pyridazinyl, triazinyl, furyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, purinyl, carbazolyl, benzimidazolyl, indolinyl, pyrrolyl, azolyl, quinolinyl, isoquinolinyl, benzisoxazolyl, imidazo[1,2-b]thiazolyl or the like. The carbon atoms or heteroatoms in the ring(s) of the heteroaryl group can be oxidized to form a carbonyl, an N-oxide, or a sulfonyl group (or other oxidized linkage) or a nitrogen atom can be quaternized, provided the aromatic nature of the ring is preserved. In some embodiments, the heteroaryl group has from 3 to 10 carbon atoms, from 3 to 8 carbon atoms, from 3 to 5 carbon atoms, from 1 to 5 carbon atoms, or from 5 to 10 carbon atoms. In some embodiments, the heteroaryl group contains 3 to 14, 4 to 12, 4 to 8, 9 to 10, or 5 to 6 ring-forming atoms. In some embodiments, the heteroaryl group has 1 to 4, 1 to 3, or 1 to 2 heteroatoms.

The compounds described herein can be asymmetric (e.g., having one or more stereocenters). All stereoisomers, such as enantiomers and diastereomers, are intended unless otherwise indicated. All stereoisomers listed as “arbitrarily assigned” are presumed and not intended to be limiting. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Geometric isomers of olefins, C═N double bonds, and the like can also be present in the compounds described herein, and all such stable isomers are contemplated in the present invention. Cis and trans geometric isomers of the compounds of the present invention may be isolated as a mixture of isomers or as separated isomeric forms.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the invention also include all isotopes of atoms occurring in the intermediates or final compounds. Isotopes include those atoms having the same atomic number but different mass numbers. For example, isotopes of hydrogen include tritium and deuterium. In some embodiments, the compounds of the invention include at least one deuterium atom.

The term, “compound,” as used herein is meant to include all stereoisomers, geometric isomers, tautomers, and isotopes of the structures depicted, unless otherwise specified.

All compounds, and pharmaceutically acceptable salts thereof, can be found together with other substances such as water and solvents (e.g., in the form of hydrates and solvates) or can be isolated.

In some embodiments, the compounds of the invention, or salts thereof, are substantially isolated. By “substantially isolated” is meant that the compound is at least partially or substantially separated from the environment in which it was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of a compound of the invention, or salt thereof.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

Prodrugs of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a drug precursor that is transformed in vivo to yield an active pharmaceutical ingredient, such as a compound as described herein, where R is H. The transformation may occur by various mechanisms (e.g., by metabolic or chemical processes). Such as, for example, through hydrolysis in blood. A discussion of the use of prodrugs is provided by T. Higuchi and W. Stella, “Pro-drugs as Novel Delivery Systems.” Vol. 14 of the A.C.S. Symposium Series, and in Bioreversible Carriers in Drug Design, ed. Edward B. Roche, American Pharmaceutical Association and Pergamon Press, 5 1987. For example, for carboxylic acid-containing compounds, a prodrug can comprise an ester formed by the replacement of the hydrogen atom of the acid group with a group such as, for example, C₁₋₈ alkyl, C₁₋₆ alkanoyloxymethyl, a 1-(alkanoyloxy)ethyl group having from 4 to 9 carbon atoms, a 1-methyl-1-(alkanoyloxy)-ethyl group having from 5 to 10 carbon atoms, a alkoxycarbonyloxymethyl group having from 3 to 6 carbon atoms, a 1-(alkoxycarbonyloxy)ethyl group having from 4 to 7 carbon atoms, a 1-methyl-1-(alkoxycarbonyloxy)ethyl group having from 5 to 8 carbon atoms, a N-(alkoxycarbonyl)aminomethyl group having from 3 to 9 carbon atoms, a 1-(N-(alkoxycarbonyl)amino)ethyl group having from 4 to 10 carbon atoms, 3-phthalidyl, 4-crotono lactonyl, gamma-butyrolacton-4-yl, di-N,N—C₁₋₄ alkylamino-C₁₋₄ alkyl (such as f-dimethylaminoethyl), carbamoyl-C₁₋₄ alkyl, N,N-di-C₁₋₄ alkylcarbamoyl-C₁₋₄ alkyl and piperidino-, pyrrolidino- or morpholino-C₁₋₄ alkyl, and the like.

### Methods of Use

One aspect of the present disclosure provides a method of inhibiting XPO1. The inhibition can be carried out in vitro and/or in vivo by contacting preparations or cells containing XPO1 with a compound of the invention, or pharmaceutically acceptable salt thereof.

Some embodiments provide methods of use of the compounds and compositions described herein for reducing tumor volume, reducing tumor growth, and/or increasing patient survival, where the patient has a tumor. In some embodiments, the tumor is characterized by overexpression of XPO1 or abnormal levels of XPO1.

Various cancers may be treated with the compounds and compositions described herein. As used herein, the term “cancer” refers to any of various malignant neoplasms characterized by the proliferation of anaplastic cells that tend to invade surrounding tissue and metastasize to new body sites and also refers to the pathological condition characterized by such malignant neoplastic growths. Cancers may be tumors or hematological malignancies, and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus.

In some embodiments, the types of carcinomas which may be treated with the compounds and compositions of the present disclosure include, but are not limited to, papilloma/carcinoma, choriocarcinoma, endodermal sinus tumor, teratoma, adenoma/adenocarcinoma, melanoma, atypical fibroxanthoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma, sinonasal undifferentiated carcinoma and urothelial carcinoma.

In some embodiments, the types of carcinomas which may be treated with the compounds and compositions of the present disclosure include, but are not limited to, soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma.

As a non-limiting example, the carcinoma which may be treated by the compounds and compositions of the present disclosure may be Acral melanoma, Acute granulocytic leukemia, Acute lymphocytic leukemia, Acute myelogenous leukemia, Adenocarcinoma, Adenosarcoma, Adrenal cancer, Adrenocortical carcinoma, Anal cancer, Anaplastic astrocytoma, Anaplastic ependymoma, Anaplastic oligodendroglioma, Anaplastic thyroid carcinoma, Angiosarcoma, Appendix cancer, Astrocytoma, Basal cell carcinoma, B-Cell lymphoma, Diffuse Large B-Cell Lymphoma (DLBCL), Bile duct cancer, Bladder cancer, Bone cancer, Bowel cancer, Benign thyroid cancer, Brain cancer, Brain stem glioma, Brain tumor, Breast cancer, Carcinoid tumors, Cervical cancer, Cholangiocarcinoma, Chondrosarcoma, Chronic lymphocytic leukemia, Chronic myelogenous leukemia, Clear cell carcinoma, Colon cancer, Colorectal cancer, Craniopharyngioma, Conjuctival melanoma, Cutaneous lymphoma, Cutaneous melanoma, Diffuse astrocytoma, Ductal carcinoma in situ, Endometrial cancer, Ependymoma, Epithelioid sarcoma, Esophageal cancer, Esophageal squamous cell carcinoma, Ewing sarcoma, Extrahepatic bile duct cancer, Eye cancer, Fallopian tube cancer, Fibrosarcoma, Gallbladder cancer, Ganglioglioma, Gastric cancer, Gastrointestinal cancer, Gastrointestinal carcinoid cancer, Gastrointestinal stromal tumors, General, Germ cell tumor, Glioblastoma multiforme, Glioma, Gliosarcoma, Hairy cell leukemia, Head and neck cancer, Head and neck squamous cell carcinoma, Hemangioendothelioma, Hepatocellular carcinoma, Hodgkin lymphoma, Hodgkin's disease, Hodgkin's lymphoma, Hypopharyngeal cancer, Infiltrating ductal carcinoma, Infiltrating lobular carcinoma, Inflammatory breast cancer, Intestinal Cancer, Intrahepatic bile duct cancer, Invasive/infiltrating breast cancer, Islet cell cancer, Jaw cancer, Kaposi sarcoma, Kidney cancer, Laryngeal cancer, Leiomyosarcoma, Leptomeningeal metastases, Leukemia, Lip cancer, Liposarcoma, Liver cancer, Lobular carcinoma in situ, Low-grade astrocytoma, Lung adenocarcinoma, Lung cancer, Lymph node cancer, Lymphoma, Male breast cancer, Malignant peripheral nerve sheath tumor, Medullary carcinoma, Medulloblastoma, Melanoma, Meningioma, Merkel cell carcinoma, Mesenchymal chondrosarcoma, Mesothelioma, Metastatic breast cancer, Metastatic melanoma, Metastatic squamous neck cancer, Mixed gliomas, Myxoid liposarcoma, Mouth cancer, Mucinous carcinoma, Mucosal melanoma, Multiple myeloma, Nasal cavity cancer, Nasopharyngeal cancer, Neck cancer, Neuroblastoma, Neurocytoma, Neuroendocrine carcinoma, Neuroendocrine tumors, Non-Hodgkin lymphoma, Non-Hodgkin's lymphoma, Non-small cell lung cancer, Oat cell cancer, Ocular cancer, Ocular melanoma, Oligoastrocytoma, Oligodendroglioma, Oral cancer, Oral cavity cancer, Oropharyngeal cancer, Osteogenic sarcoma, Osteosarcoma, Ovarian cancer, Ovarian epithelial cancer, Ovarian germ cell tumor, Ovarian primary peritoneal carcinoma, Ovarian sex cord stromal tumor, Paget's disease, Pancreatic cancer, Papillary carcinoma, Paranasal sinus cancer, Parathyroid cancer, Pelvic cancer, Penile cancer, Peripheral nerve cancer, Peritoneal cancer, Pharyngeal cancer, Phaeochromocytoma, Pilocytic astrocytoma, Pineal region tumor, Pineoblastoma, Pituitary gland cancer, Primary central nervous system lymphoma, Pleomorphic dermal sarcoma, Pleomorphic xanthoastrocytoma, Poorly differentiated thyroid carcinoma, Prostate cancer, Rectal cancer, Renal cell cancer, Renal pelvis cancer, Rhabdomyosarcoma, Salivary gland cancer, Sarcoma, Sarcoma, bone, Sarcoma, soft tissue, Sarcoma, uterine, Serous carcinoma, Sinus cancer, Sino nasal malignant melanoma Skin cancer, Small cell lung cancer, Small intestine cancer, Soft tissue sarcoma, Solitary Fibrous tumor Spinal cancer, Spinal column cancer, Spinal cord cancer, Spinal tumor, Spitzoid neoplasm, Squamous cell carcinoma, Squamous cell carcinoma of the cervix, Stomach cancer, Synovial sarcoma, T-cell lymphoma, Tall cell papillary thyroid carcinoma, Testicular cancer, Testicular germ cell tumor, Throat cancer, Thymoma/thymic carcinoma, Thyroid cancer, Thyroid carcinoma, Tongue cancer, Tonsil cancer, Transitional cell cancer, Transitional cell cancer, Transitional cell cancer, Triple-negative breast cancer, Tubal cancer, Tubular carcinoma, Ureteral cancer, Urethral cancer, Uterine adenocarcinoma, Uterine cancer, Uterine sarcoma, Vaginal cancer, and Vulvar cancer.

In some embodiments, compounds and compositions of the present disclosure may be used to treat central nervous system (CNS) tumors, such as but not limited to brain tumor, spinal cord tumor, glioblastoma, meningioma, medulloblastomas, craniopharyngioma, astrocytic tumors, oligodendroglial tumors, mixed gliomas, ependymal tumors, pineal parenchymal tumors, meningeal tumors, or germ cell tumors.

In some embodiments, compounds and compositions of the present disclosure may be used to treat hepatocellular carcinoma (HCC).

In some embodiments, the compounds and compositions of the present disclosure may be used to treat hematological cancers such as leukemia and lymphoma. In some embodiments, the hematological cancer is selected from Acute lymphoblastic leukemia (ALL), Acute myelogenous leukemia (AML), Chronic myelogenous leukemia (CML), Chronic lymphocytic leukemia (CLL), Hairy cell leukemia, Hodgkin's disease (four subtypes), Mixed-phenotype acute leukemia (MPAL), Non-Hodgkin lymphoma (many subtypes), Multiple myeloma (MM), refractory multiple myeloma (RMM), Myelodysplastic syndrome (MDS), Myelofibrosis (e.g., primary myelofibrosis), Myeloproliferative disease, Polycythemia vera (PV), Essential thrombocytosis (ET), and Amyloid due to light-chain disease.

In some embodiments, the present invention provides a method of treating a solid tumor comprising administering a therapeutically effective amount of a compound of Formula I, or a pharmaceutically acceptable salt thereof. In some embodiments, the solid tumor is selected from bladder cancer, bone cancer, brain cancer, breast cancer, cervical cancer, CNS cancers, colon cancer, colorectal cancer, endometrial cancer, esophogeal cancer, gastric cancer, head and neck cancer, hepatocellular carcinoma, kidney cancer, liver cancer, lung cancer, melanoma, neuroendocrine tumors, ovarian cancer, pancreatic cancer, prostate cancer, rectal cancer, soft tissue sarcoma, spinal tumor, testicular cancer, and uterine cancer. In some embodiments, the solid tumor is selected from CNS cancers, colon cancer, lung cancer, prostate cancer, breast cancer, liver cancer, and endometrial cancer.

The compounds and compositions disclosed herein may be useful to treat various other diseases, conditions or disorders associated with activity of XPO1. In some embodiments, compounds and compositions of the present disclosure may be used to treat an inflammatory disorder. In some embodiments, the inflammatory disorder is myelitis, neuroinflammation, inflammatory bowel disease, dermatitis, or rheumatoid arthritis. In some embodiments, the inflammatory bowel disease is ulcerative colitis or Crohn's disease.

In some embodiments, compounds and compositions of the present disclosure may be used to treat an autoimmune disease. In some embodiments, the autoimmune disease is Celiac disease or rheumatoid arthritis.

In some embodiments, compounds and compositions of the present disclosure may be used to treat a neurodegenerative disease. In some embodiments, the neurodegenerative disease is Parkinson's disease, Alzheimer's disease, Huntington's disease, multiple sclerosis, or Amyotrophic lateral sclerosis (ALS).

In some embodiments, compounds and compositions of the present disclosure may be used to treat an infectious disease. In some embodiments, the infectious disease is HIV-1, a SARS-CoV-2 infection (i.e., COVID-19), a respiratory syncytial virus (RSV) infection, or influenza.

Combination Therapies In some embodiments, the present invention provides a method of treating a disease or disorder described herein, comprising administering a compound of the present disclosure in combination with one or more additional active agents or therapies. Suitable pharmaceutical agents or therapies that may be used in combination with the compounds of the present disclosure include anti-cancer agents, immune enhancers, immunosuppressants, immunotherapies, radiation, anti-tumor and anti-viral vaccines, cytokine therapy (e.g., IL2, GM-CSF, etc.), and/or kinase (tyrosine or serine/threonine), epigenetic or signal transduction inhibitors.

The compounds of the present disclosure and the additional active agent(s) may be administered simultaneously, sequentially, or at any order. The compounds of the present disclosure and the additional active agent(s) may be administered at different dosages, with different dosing frequencies, or via different routes, whichever is suitable.

Suitable agents for use in combination with the compounds of the present invention for the treatment of cancer include chemotherapeutic agents, targeted cancer therapies, immunotherapies or radiation therapy. Compounds of this invention may be effective in combination with anti-hormonal agents for treatment of breast cancer and other tumors. Suitable examples are anti-estrogen agents including but not limited to tamoxifen and toremifene, aromatase inhibitors including but not limited to letrozole, anastrozole, and exemestane, adrenocorticosteroids (e.g. prednisone), progestins (e.g. megastrol acetate), and estrogen receptor antagonists (e.g. fulvestrant). Suitable anti-hormone agents used for treatment of prostate and other cancers may also be combined with compounds of the present invention. These include anti-androgens including but not limited to flutamide, bicalutamide, and nilutamide, luteinizing hormone-releasing hormone (LHRH) analogs including leuprolide, goserelin, triptorelin, and histrelin, LHRH antagonists (e.g. degarelix), androgen receptor blockers (e.g. enzalutamide) and agents that inhibit androgen production (e.g. abiraterone).

Angiogenesis inhibitors may be efficacious in some tumors in combination with FGFR inhibitors. These include antibodies against VEGF or VEGFR or kinase inhibitors of VEGFR. Antibodies or other therapeutic proteins against VEGF include bevacizumab and aflibercept. Inhibitors of VEGFR kinases and other anti-angiogenesis inhibitors include but are not limited to sunitinib, sorafenib, axitinib, cediranib, pazopanib, regorafenib, brivanib, and vandetanib.

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (Cytoxan™), ifosfamide, melphalan, chlorambucil, pipobroman, triethylene-melamine, triethylenethio¬phosphoramine, busulfan, carmustine, lomustine, streptozocin, dacarbazine, and temozolomide.

Other anti-cancer agent(s) include antibody therapeutics to checkpoint or costimulatory molecules such as CTLA-4, PD-1, PD-L1 or 4-1BB, respectively, or antibodies to cytokines (IL-10, TGF-α, etc.). Exemplary cancer immunotherapy antibodies include pembrolizumab, ipilimumab, nivolumab, atezolizumab and durvalumab. Additional anti-cancer agent(s) include antibody therapeutics directed to surface molecules of hematological cancers such as ofatumumab, rituximab and alemtuzumab.

In some embodiments, the additional active agents may be anti-cancer agents, such as but not limited to PARP inhibitors, ATR inhibitors, ATM inhibitors, CHK1/CHK2 inhibitors, wee 1 inhibitors, CDK4/6 inhibitors, antiapoptotic inhibitors, BCL-XL inhibitors, kinase inhibitors, PI3K inhibitors, DNA damaging agents (including but not limited to radiation agents), or platinum therapies. In some embodiments, the additional active agent is Temozolomide (TMZ), Lomustine (CCNU), Val-083, BGB-290 (PARP inhibitor), Olaparib (PARP inhibitor), BAY 1895344 (ATR inhibitor), AZD6738 (ATR inhibitor), AZD1390 (ATM inhibitor), AZD7762 (CHK1/CHK2 inhibitor), AZD1775 (wee 1 inhibitor), Palbociclib (CDK4/6 inhibitor), A-1155463 (BCL-XL inhibitor) and A-1331852 (BCL-XL inhibitor), Regorafenib, or Paxalisib, at varying concentrations.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” a cell with a compound of the invention includes the administration of a compound of the present invention to an individual or patient, such as a human, having, for example, cells expressing or comprising XPO1. The term “contacting” can also include introducing a compound of the invention into a sample containing a cellular or preparation containing cells.

As used herein, the term “individual” or “patient,” used interchangeably, refers to mammals, and particularly humans. The individual or patient can be in need of treatment.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease in an individual who is experiencing or displaying the pathology or symptomatology of the disease (i.e., arresting further development of the pathology and/or symptomatology), or 2) ameliorating the disease in an individual who is experiencing or displaying the pathology or symptomatology of the disease (i.e., reversing the pathology and/or symptomatology).

As used herein the term “preventing” or “prevention” refers to preventing the disease in an individual who may be predisposed to the disease but does not yet experience or display the pathology or symptomatology of the disease. In some embodiments, the invention is directed to a method of preventing a disease in a patient, by administering to the patient a therapeutically effective amount of a compound of the invention, or a pharmaceutically acceptable salt thereof.

### Pharmaceutical Formulations and Dosage Forms

When employed as pharmaceuticals, the compounds of the invention can be administered in the form of pharmaceutical compositions. A pharmaceutical composition refers to a combination of a compound of the invention, or its pharmaceutically acceptable salt, and at least one pharmaceutically acceptable carrier. These compositions can be prepared in a manner well known in the pharmaceutical art, and can be administered by a variety of routes, depending upon whether local or systemic treatment is desired and upon the area to be treated. Administration may be oral, topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, or parenteral.

This invention also includes pharmaceutical compositions which contain, as the active ingredient, one or more of the compounds of the invention above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

The compositions can be formulated in a unit dosage form. The term “unit dosage form” refers to a physically discrete unit suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid pre-formulation composition containing a homogeneous mixture of a compound of the present invention. When referring to these pre-formulation compositions as homogeneous, the active ingredient is typically dispersed evenly throughout the composition so that the composition can be readily subdivided into equally effective unit dosage forms such as tablets, pills and capsules. This solid pre-formulation is then subdivided into unit dosage forms of the type described above containing from, for example, 0.1 to about 500 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. Effective doses can be extrapolated from dose-response curves derived from in vitro or animal model test systems.

### Kits

The disclosure provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

The kit may further comprise packaging and instructions and/or a delivery agent to form a formulation, e.g., for administration to a subject in need of treatment using the compositions described herein. The delivery agent may comprise a saline, a buffered solution, a lipidoid, a dendrimer or any suitable delivery agent.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples were found to be inhibitors of XPO1 according to one or more of the assays provided herein.

### Instruments

### Lcms Conditions:

Instrument name: Agilent Technologies 1290 infinity 11. Method A: Method: A-0.1% trifluoroacetic acid (TFA) in H₂O, B-0.1% TFA in acetonitrile (ACN); flow rate: 2.0 mL/min; column: XBridge C8 (50×4.6 mm, 3.5 μm) or BEH C8, +ve mode. Method B: Method: A-10 mM NH₄HCO₃ in H₂O, B-ACN; flow rate: 1.0 mL/min; column: XBridge C8 (50×4.6 mm, 3.5 μm) or BEH C8, +ve mode. Method C: Method: A-0.1% HCOOH in H₂O, B-0.1% formic acid (FA) in ACN; flow rate: 1.5 mL/min; column: ZORBAX XDB C-18 (50×4.6 mm, 3.5 μm) or BEH C8, +ve mode. Method D: Method: A-10 mM NH₄OAc in H₂O, B-ACN; flow rate: 1.0 mL/min; column: XBridge C8 (50×4.6 mm, 3.5 μm) or BEH C8, +ve mode.

Instrument name: Agilent 1260 infinity II. Method E: Method: A-0.1% NH₃·H₂O in H₂O, B-ACN; flow rate: 2.0 mL/min; column: XBridge C18 (50×4.6 mm, 3.5 μm), ESI mode. Method F: Method: A-0.05% FA in H₂O, B-0.05% FA in ACN; flow rate: 2.0 mL/min; column: Welch Boltimate EXT C18 core-shell (4.6*50 mm, 2.7 μm). ESI mode.

Instrument name: Agilent 1260-6120B QuaMS. Method G: Method: A-0.05% TFA in H₂O, B-ACN; flow rate: 2.0 mL/min; Column: Welch Boltimate C18 core-shell (4.6*50 mm, 2.7 μm). ESI mode.

Instrument name: waters UPLC H-Class. Method H: Method: A-0.05% FA in H₂O, B-0.05% FA in ACN; flow rate: 0.6 mL/min; column: Waters UPLC BEH C18 (2.1 mm*50 mm 1.7 μm). ESI mode.

### HPLC Analysis Conditions:

Instrument name: Agilent 1200 Series instruments as followed using % with UV detection (maxplot). Method A: Method: A-0.1% TFA in H₂O, B-0.1% TFA in ACN; flow rate: 2.0 mL/min; column: XBridge C8 (50×4.6 mm, 3.5 μm). Method B: Method: A-10 mM NH₄HCO₃ in H₂O, B-ACN; flow rate: 1.0 mL/min; column: XBridge C8 (50×4.6 mm, 3.5 μm).

Instrument name: Waters UPLC as followed using % with UV detection (maxplot). Method C: Method: A-0.02% TFA in H₂O, B-0.02% TFA in ACN; flow rate: 2.0 mL/min; column: ACQUITY UPLC BEH C18 (2.1*150 mm, 1.7 μm). Method D: Method: A-10 mM NH₄HCO₃ in H₂O, B-ACN; flow rate: 1.0 mL/min; column: YMC Triart C18 (4.6*150 mm, 3 μm). Method E: Method: A-0.02% TFA in H₂O, B-0.02% TFA in ACN; flow rate: 0.4 mL/min; column: Welch Ultimate UHPLC LP-C18 (2.1*100 mm, 1.8 μm).

### Prep-HPLC Purification Conditions:

Method A: A-0.1% TFA in H₂O, B-MeOH or ACN; column: Sunfire C8 (19×250 mm, 5 μm) or Sunfire C18 (30×250 mm, 10 μm). Method B: A-10 mM NH₄HCO₃ in H₂O, B-MeOH or ACN, Column: Sunfire C8 (19×250 mm, 5 μm) or Sunfire C18 (30×250 mm, 10 μm). Method C: A-0.1% FA in H₂O, B-ACN; column: Welch Ultimate XB-C18 (21.2*150 mm Sum) or (50*150 mm, 5 μm). Method D: A-0.1% NH₃H₂O/H₂O, B-ACN, column: Xbridge C18 (19*250 mm 5 μm). Method E: A-0.05% TFA in H₂O, B-CAN; column: Waters SunFire C18 OBD (19*150 mm 5 μm).

### CHIRAL SFC Purification Conditions:

Instrument name: PIC SFC 175. Method A: Mobile Phase: 0.1% Isopropylamine in IPA:MeOH (1:1), flow rate: 100 mL/min; column: Lux A1 (250×30 mm, 5 μm); Method B: Mobile Phase: 0.1% Isopropylamine in IPA:MeOH (1:1), flow rate: 100 mL/min; column: Chiralpak OX-H (250×30 mm, 5 μm); Method C: Mobile Phase: 0.5% Isopropylamine in IPA:MeOH (1:1), flow rate: 100 mL/min; column: Lux A1 (250×30 mm, 5 μm).

Instrument name: SFC-150mgm (Waters); Method D: Mobile phase: CO₂/EtOH[0.5% NH3(7M in MeOH)]=90/10, Flow rate: 100 mL/min; Column: Daicel OJ (25*250 mm, 10 μm); Method E: Mobile phase: CO₂/MeOH[0.2% NH₃(7M in MeOH)]=65/35, Flow rate: 100 mL/min; column: YMC Cellulose-SC (25*250 mm, 5 μm); Method F: Mobile phase: CO₂/MeOH[0.2% NH₃(7M in MeOH)]=90/10; Flow rate: 120 mL/min; Daicel OJ-3 (25*250 mm, 10 μm); Method G: Mobile phase: CO₂/MeOH[0.2% NH₃(7M in MeOH)]=70/30, Flow rate: 100 mL/min; column: YMC Cellulose-SC (20*250 mm, 5 μm)

Instrument name: Waters 80Q. Method H: Mobile Phase: CO₂:MeOH (0.1% NH₃H₂O)=80:20, flow rate: 60 mL/min; column: OJ-H (30*250 mm, 5 μm); Method I: Mobile Phase: CO₂:MeOH (0.1% NH₃H₂O)=80:20, flow rate: 50 mL/min; column: AD-H (30*250 mm, 5 μm).

NMR instrument name: BRUKER NMR, model AV-II, AV-III and AV-NEO 400 MHz FT-NMR.

### Example 1. Synthesis of 2-5-cyclohexyl-1-(4-(trifluoromethyl)benzyl)piperidin-3-yl)acetic acid (Compound 11)

2-5-cyclohexyl-1-(4-(trifluoromethyl)benzyl)piperidin-3-yl)acetic acid (Compound 11) was prepared according to the scheme below.

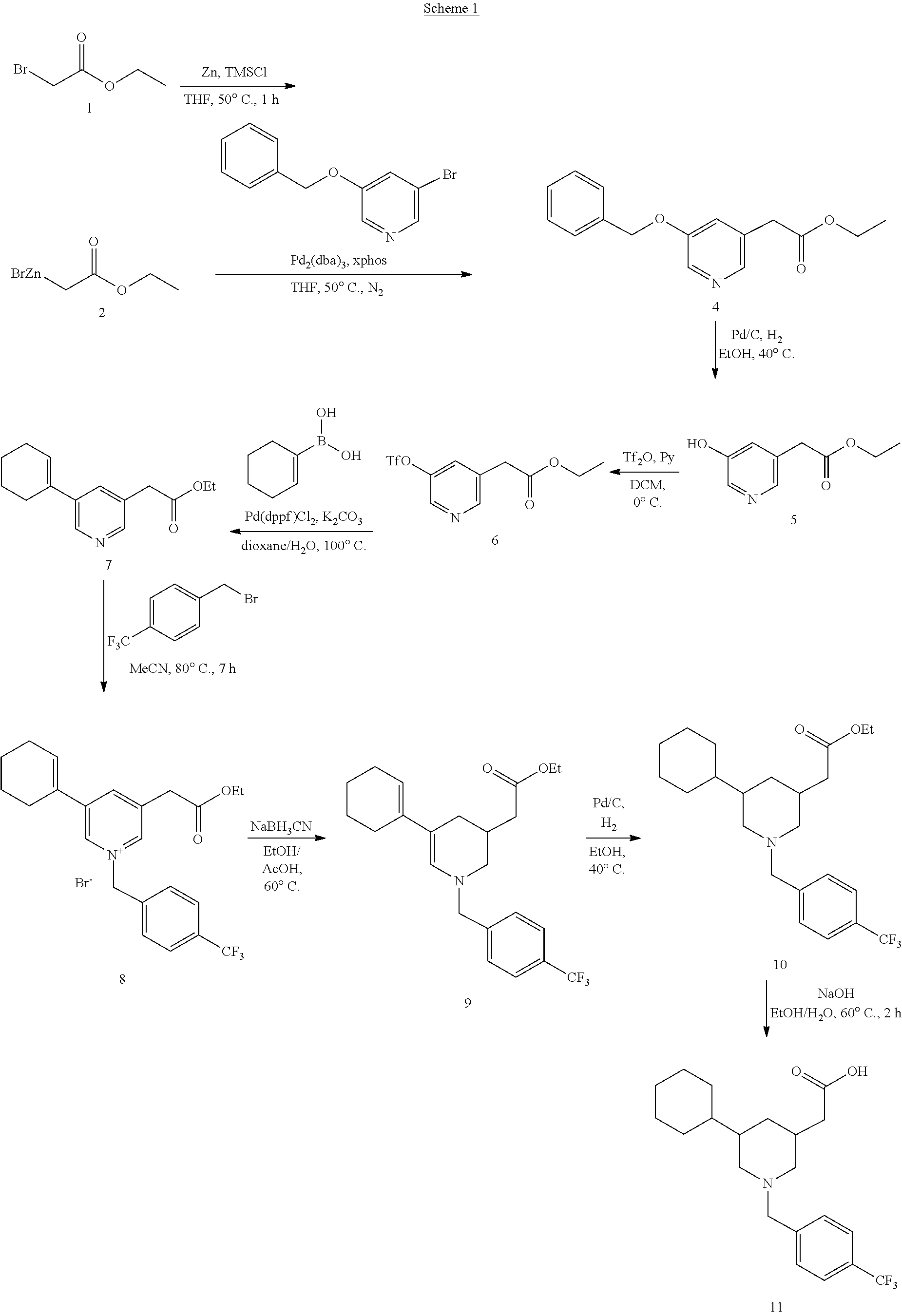

### Step 1: (2-ethoxy-2-oxoethyl)zinc(H) bromide (Compound 2)

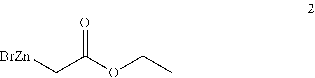

To a mixture of zinc powder (35.0 g, 539 mmol) in dry THE (360 mL) was added TMSCl (3.90 g, 35.9 mmol). After the reaction mixture was stirred at 50° C. under N₂ atmosphere for 0.5 h, ethyl 2-bromoacetate (Compound 1, 60.0 g, 359 mmol) was added. The reaction mixture was stirred at 50° C. under N₂ atmosphere for 1.0 h. The light yellow solution was used for subsequent experiments directly.

### Step 2: ethyl 2-(5-(benzyloxy)pyridin-3-yl)acetate (Compound 4)

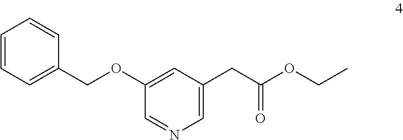 [SMILES: CCOC(=O)Cc1cncc(OCc2ccccc2)c1]

To a solution of 3-(benzyloxy)-5-bromopyridine (29.9 g, 113 mmol), Pd₂(dba)₃ (2.07 g, 2.26 mmol), and Xphos (2.15 g, 4.52 mmol) in THE was added 2-ethoxy-2-oxoethyl)zinc(II) bromide (Compound 2, 1 M in THF, 340 mL). The reaction mixture was stirred at 60° C. under N₂. After completion (monitored by TLC and LC-MS), the reaction mixture was poured into ice water. The mixture was filtered by diatomite and the filtrate was extracted with EtOAc (3×500 mL). The combined organic layers were washed with brine (2×300 mL), dried over anhydrous sodium sulfate, filtered, and concentrated. The residue was purified by flash chromatography on silica gel to give ethyl 2-(5-(benzyloxy)pyridin-3-yl)acetate (Compound 4). Yield: crude (20.3 g, yellow oil). LCMS: (Method F) 272.1 (M+H).

### Step 3: ethyl 2-(5-hydroxypyridin-3-yl)acetate (Compound 5)

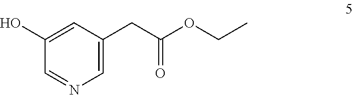

A mixture of ethyl 2-(5-(benzyloxy)pyridin-3-yl)acetate (Compound 4, crude, 20.3 g, 74.9 mmol) and Pd/C (10 wt %, 7.93 g, 7.49 mmol) in EtOH (150 mL) was stirred at 40° C. under N₂ atmosphere. After completion (monitored by TLC and LC-MS), the reaction mixture was filtered and the filtrate was concentrated under reduced pressure. The residue was purified by flash chromatography on silica gel to give ethyl 2-(5-hydroxypyridin-3-yl)acetate (Compound 5). Yield: crude (12.0 g, yellow oil). LCMS: (Method F) 182.1 (M+H).

### Step 4: ethyl 2-(5-(((trifluoromethyl)sulfonyl)oxy)pyridin-3-yl)acetate (Compound 6)

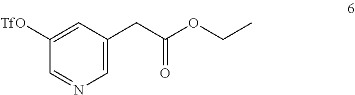 [SMILES: CCOC(=O)Cc1cncc(OS(=O)(=O)C(F)(F)F)c1]

To a solution of ethyl 2-(5-hydroxypyridin-3-yl)acetate (Compound 5, 10.0 g, 55.2 mmol) and pyridine (13.1 g, 166 mmol) in DCM (120 mL) was added Tf₂O (17.1 g, 60.7 mmol) at 0° C. The reaction mixture was stirred at room temperature. After completion (monitored by TLC and LC-MS), the reaction mixture was washed with brine (2×20 mL), dried over anhydrous Na₂SO₄, filtered, and concentrated. The residue was purified by flash chromatography on silica gel to give ethyl 2-(5-(((trifluoromethyl)sulfonyl)oxy)pyridin-3-yl)acetate (Compound 6). Yield: 40.5% (7.00 g, yellow oil). LCMS: (Method F) 314.0 (M+H).

### Step 5: ethyl 2-(5-(cyclohex-1-en-1-yl)pyridin-3-yl)acetate (Compound 7)

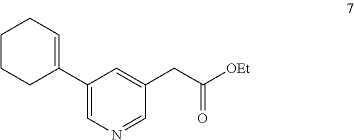 [SMILES: CCOC(=O)Cc1cncc(C2=CCCCC2)c1]

A solution of ethyl 2-(5-(((trifluoromethyl)sulfonyl)oxy)pyridin-3-yl)acetate (Compound 6, 700 mg, 2.23 mmol), cyclohex-1-en-1-ylboronic acid (310 mg, 2.46 mmol), Pd(dppf)Cl₂ (23.0 mg, 0.032 mmol), and K₂CO₃ (440 mg, 3.19 mmol) in dioxane/H₂O (10 mL/2 mL) was stirred at 100° C. under N₂ atmosphere. After completion (monitored by LC-MS), organic solvent was removed under vacuum and the residue was extracted with EtOAc (3×10 mL). The combined organic layers were washed with brine (2×10 mL), dried over anhydrous sodium sulfate, filtered, and concentrated. The residue was purified by flash chromatography on silica gel (PE/EA=20:1-5:1) to give ethyl 2-(5-(cyclohex-1-en-1-yl)pyridin-3-yl)acetate (Compound 7). Yield: 81% (489 mg, yellow oil). LCMS: (Method F) 246.1 (M+H).

### Step 6: 3-(cyclohex-1-en-1-yl)-5-(2-ethoxy-2-oxoethyl)-1-(4-(trifluoromethyl)benzyl) pyridin-1-ium bromide (Compound 8)

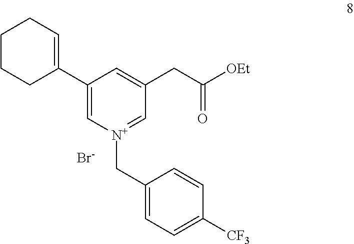 [SMILES: CCOC(=O)Cc1cc(C2=CCCCC2)c[n+](Cc2ccc(C(F)(F)F)cc2)c1 ; [Br-]]

A solution of ethyl 2-(5-(cyclohex-1-en-1-yl)pyridin-3-yl)acetate (Compound 7, 480 mg, 1.96 mmol) and 1-(bromomethyl)-4-(trifluoromethyl)benzene (561 mg, 2.35 mmol) in MeCN (5 mL) was stirred at 80° C. for 7 hours. After completion (monitored by TLC), the reaction mixture was concentrated under vacuum and the residue was purified by flash chromatography on silica gel (DCM/MeOH=30:1-5:1) to give 3-(cyclohex-1-en-1-yl)-5-(2-ethoxy-2-oxoethyl)-1-(4-(trifluoromethyl)benzyl) pyridin-1-ium bromide (Compound 8). Yield: 79% (748 mg, light yellow oil).

### Step 7: ethyl 2-(5-(cyclohex-1-en-1-yl)-1-(4-(trifluoromethyl)benzyl)-1,2,3,4-tetrahy-dropyridin-3-yl)acetate (Compound 9)

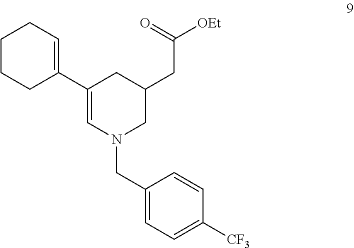 [SMILES: CCOC(=O)CC1CC(C2=CCCCC2)=CN(Cc2ccc(C(F)(F)F)cc2)C1]

To a solution of 3-(cyclohex-1-en-1-yl)-5-(2-ethoxy-2-oxoethyl)-1-(4-(trifluoromethyl)benzyl)pyridin-1-ium bromide (Compound 8, 500 mg, 1.03 mmol) in EtOH/AcOH (10 mL/1 mL) was added NaBH₃CN (649 mg, 10.3 mmol) in batches at room temperature. The reaction mixture was stirred at 60° C. After completion (monitored by LC-MS), the reaction mixture was neutralized with 10% aq. Na₂CO₃ solution. The mixture was extracted with dichloromethane (DCM, 3×10 mL). The combined organic layers were washed with brine (2×10 mL), dried over anhydrous sodium sulfate, filtered, and concentrated to get the crude product. Yield: crude (498 mg, yellow oil). LCMS: (Method F) 410.2 (M+H).

### Step 8: ethyl 2-(5-cyclohexyl-1-(4-(trifluoromethyl)benzyl)piperidin-3-yl) acetate (Compound 10)

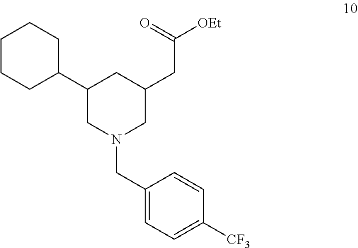 [SMILES: CCOC(=O)CC1CC(C2CCCCC2)CN(Cc2ccc(C(F)(F)F)cc2)C1]

A mixture of ethyl 2-(5-(cyclohex-1-en-1-yl)-1-(4-(trifluoromethyl)benzyl)-1,2,3,4-tetrahy-dropyridin-3-yl) acetate (Compound 9, crude, 498 mg, 1.23 mmol) and Pd/C (10 wt %, 390 mg) in EtOH (10 mL) was stirred under H₂ at 40° C. After completion (monitored by LCMS), the reaction mixture was filtered, and the filtrate was concentrated under vacuum. The residue was purified by prep-HPLC (Method C) to give a first-eluting isomer and a second-eluting isomer.

First-eluting isomer HPLC (minor isomer): LCMS: (Method F) 412.5 (M+H), Rt. 1.78 min.

Second-eluting isomer HPLC (major isomer): Yield: 6.9% (35 mg, white solid). LCMS: (Method F) 412.5 (M+H), Rt. 1.93 min, 95.28% (Max).

### Step 9: 2-(-5-cyclohexyl-1-(4-(trifluoromethyl)benzyl)piperidin-3-yl)acetic acid (Compound 11)

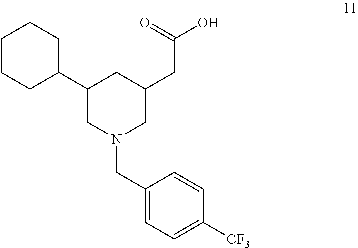 [SMILES: O=C(O)CC1CC(C2CCCCC2)CN(Cc2ccc(C(F)(F)F)cc2)C1]

A solution of the second-eluting isomer 2-(5-cyclohexyl-1-(4-(trifluoromethyl)benzyl) piperidin-3-yl) acetate (Compound 10, 21.0 mg, 0.050 mmol) and NaOH (12.0 mg, 0.31 mmol) in EtOH/H₂O (2.5 mL/0.5 mL) was stirred at 60° C. for 2 hours. After completion (monitored by LC-MS), EtOH was removed under vacuum. The residue was acidified with aq. HCl (1 N) to pH about 6-7. The resulting suspension was extracted with DCM (2×1 mL). The combined organic layer was washed with brine (2×2 mL), dried over anhydrous sodium sulfate and concentrated under vacuum. The residue was purified by prep-HPLC (Method D) to give Compound 11 as stereoisomer 1. Yield: 11% (2.2 mg, white solid). ¹H NMR (400 MHz, CDCl₃) δ 7.63 (d, J=8.0 Hz, 2H), 7.51 (d, J=8.0 Hz, 2H), 3.78-3.72 (m, 2H), 3.00 (d, J=9.6 Hz, 1H), 2.92 (d, J=10.8 Hz, 1H), 2.73 (dd, J=16.4, 6.4 Hz, 1H), 2.57 (dd, J=16.4, 5.2 Hz, 1H), 2.47-2.45 (m, 1H), 2.31-2.29 (m, 1H), 2.03 (t, J=10.8 Hz, 1H), 1.80-1.57 (m, 7H), 1.42-1.32 (m, 1H), 1.23-1.05 (m, 4H), 0.97-0.84 (m, 2H). LCMS: (Method C) 384.2 (M+H), Rt. 1.38 min, 100.00% (Max). HPLC: (Method C) Rt. 5.26 min, 99.33% (Max). Stereoisomer 1 of Compound 11 was arbitrarily assigned as a racemic mixture of trans enantiomers (e.g., 2-((3S, 5S)-5-cyclohexyl-1-(4-(trifluoromethyl)benzyl)piperidin-3-yl)acetic acid and 2-((3R, 5R)-5-cyclohexyl-1-(4-(trifluoromethyl)benzyl)piperidin-3-yl)acetic acid).

The above step was conducted with the first-eluting isomer of Compound 10 to give Compound 11 as stereoisomer 2. ¹H NMR (400 MHz, DMSO) δ 7.71 (d, J=8.0 Hz, 2H), 7.61 (d, J=8.0 Hz, 2H), 3.98 (dd, J=12.0, 20.0 Hz, 2H), 3.18 (d, J=8.0 Hz, 2H), 2.18-2.06 (m, 5H), 1.89 (d, J=8.0 Hz, 1H), 1.74-1.60 (m, 6H), 1.26-1.17 (m, 4H), 1.14-1.01 (m, 2H), 0.99-0.80 (m, 1H). LCMS: Method: A-0.1% NH₃·H₂O in H2O, B-ACN; flow rate: 2.0 mL/min; column: XBridge C18 (50×4.6 mm, 3.5 μm), ESI mode; 384.2 (M+H); Rt: 1.36 min. Stereoisomer 2 of Compound 11 was arbitrarily assigned as a racemic mixture of cis enantiomers (e.g., 2-((3R,5S)-5-cyclohexyl-1-(4-(trifluoromethyl)benzyl)piperidin-3-yl)acetic acid and 2-((3S,5R)-5-cyclohexyl-1-(4-(trifluoromethyl)benzyl)piperidin-3-yl)acetic acid).

### Example 2. Synthesis of 2-5-cycloheptyl-1-(4-(trifluoromethyl)benzyl)piperidin-3-yl)acetic acid (Compound 16)

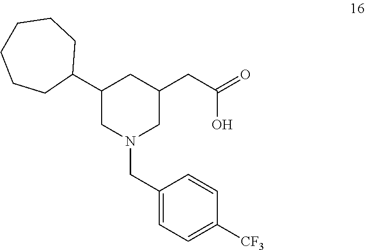 [SMILES: O=C(O)CC1CC(C2CCCCCC2)CN(Cc2ccc(C(F)(F)F)cc2)C1]

### Step 1: ethyl 2-(5-(cyclohept-1-en-1-yl)pyridin-3-yl)acetate (Compound 12)

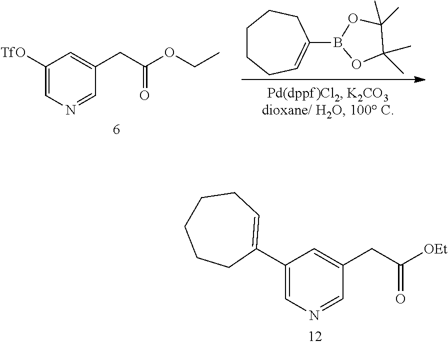

A mixture of ethyl 2-(5-(((trifluoromethyl)sulfonyl)oxy)pyridin-3-yl)acetate (Compound 6, 600 mg, 1.92 mmol), 2-(cyclohept-1-en-1-yl)-4,4,5,5-tetramethyl-1,3,2-dioxaborolane (5.33 g, 24.0 mmol), Pd(dppf)Cl₂ (1.75 g, 2.4 mmol), K₂CO₃ (6.63 g, 48.0 mmol), dioxane (50 mL) and H₂O (10 mL) was stirred at 100° C. under N₂ atmosphere. After completion (monitored by LC-MS), organic solvent was removed under vacuum and the residue was diluted with H₂O (10 mL), extracted with EtOAc (3×25 mL). The combined organic layers were washed with brine (2×30 mL), dried over anhydrous sodium sulfate, filtered, and concentrated. The residue was purified by flash chromatography on silica gel (PE/EA=20:1-8:1) to give ethyl 2-(5-(cyclohept-1-en-1-yl)pyridin-3-yl)acetate (Compound 12). Yield: 64% (4.0 g, yellow oil).

### Step 2: 3-(cyclohept-1-en-1-yl)-5-(2-ethoxy-2-oxoethyl)-1-(4-(trifluoromethyl)benzyl)pyridin-1-ium bromide (Compound 13)

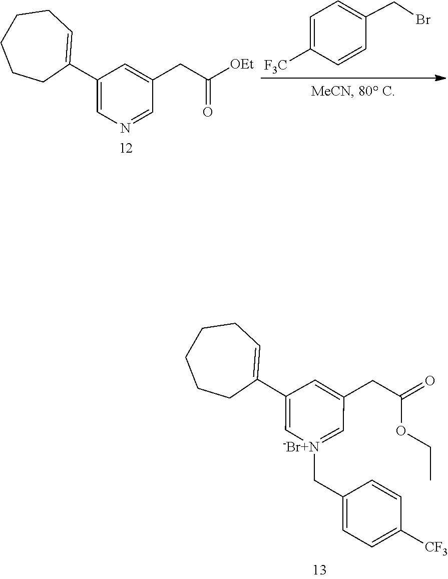

A solution of Compound 12 (4.0 g, 15.4 mmol) and 1-(bromomethyl)-4-(trifluoromethyl)benzene (4.4 g, 18.49 mmol) in MeCN (50 mL) was stirred at 80° C. for 7 hours. After completion (monitored by TLC), the reaction mixture was concentrated under vacuum and the residue was purified by flash chromatography on silica gel (DCM/MeOH=30:1-5:1) to give 3-(cyclohept-1-en-1-yl)-5-(2-ethoxy-2-oxoethyl)-1-(4-(trifluoromethyl)benzyl)pyridin-1-ium bromide (Compound 13). Yield: 91% (6.4 g, light yellow oil).

### Step 3: ethyl 2-(5-(cyclohept-1-en-1-yl)-1-(4-(trifluoromethyl)benzyl)-1,2,3,4-tetrahydropyridin-3-yl)acetate (Compound 14)

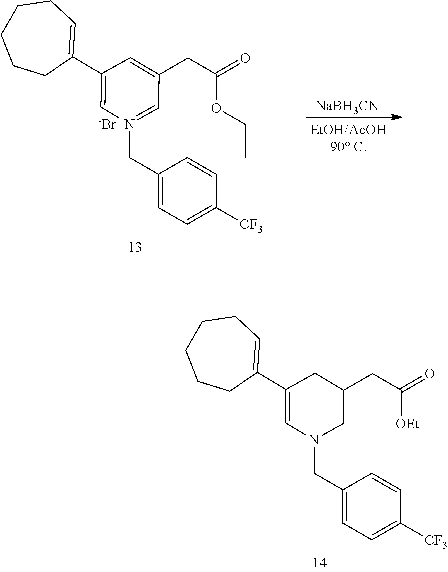

To a solution of 3-(cyclohept-1-en-1-yl)-5-(2-ethoxy-2-oxoethyl)-1-(4-(trifluoromethyl)benzyl)pyridin-1-ium bromide (Compound 13, 6.2 g, 12.4 mmol) in EtOH/AcOH (100 mL/10 mL) was added NaBH₃CN (8.1 g, 128.6 mmol) in batches at room temperature. The reaction mixture was stirred at 60° C. After completion (monitored by LC-MS), the reaction mixture was neutralized with 10% sodium carbonate aqueous solution. The mixture was extracted with dichloromethane (3×100 mL). The combined organic layers were washed with brine (2×50 mL), dried over anhydrous sodium sulfate, filtered, and concentrated to get the crude product. Yield: crude (6.9 g, yellow oil).

### Step 4: ethyl 2-(5-cycloheptyl-1-(4-(trifluoromethyl)benzyl)piperidin-3-yl)acetate (racemic mixture of cis and trans)

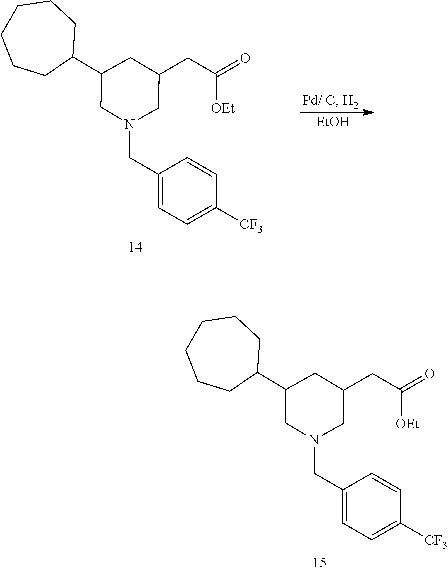 [SMILES: "EtOH" ; CCOC(=O)CC1CC(C2CCCCCC2)CN(Cc2ccc(C(F)(F)F)cc2)C1 ; "Pd/ C, H2" ; CCOC(=O)CC1CC(C2CCCCCC2)CN(Cc2ccc(C(F)(F)F)cc2)C1]

A mixture of Compound 14 (crude, 6.9 g, 16.4 mmol) and Pd/C (10 wt %, 600 mg) in EtOH (200 mL) was stirred under 15 psi H₂ at 80° C. for overnight. After completion (monitored by LCMS), the reaction mixture was filtered, and the filtrate was concentrated under vacuum. The residue was purified by reverse phase column to give ethyl 2-(5-cycloheptyl-1-(4-(trifluoromethyl)benzyl)piperidin-3-yl)acetate (Compound 15). Yield: 87% (6 g, white solid).

### Step 5: 2-(5-cycloheptyl-1-(4-(trifluoromethyl)benzyl)piperidin-3-yl)acetic acid (Compound 16)

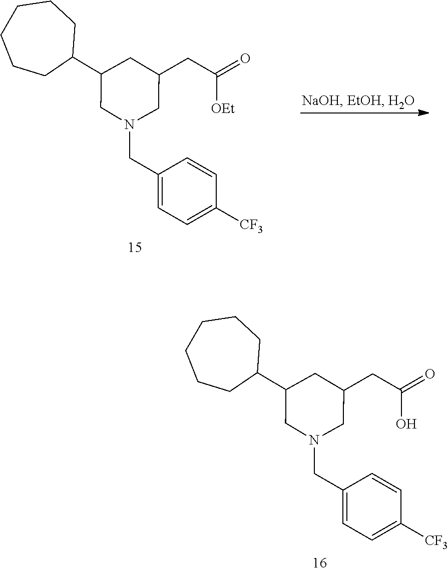

A solution of the ethyl 2[5-Cycloheptyl-1-(4-trifluoromethyl-benzyl)-piperidin-3-yl]-acetic acid ethyl ester (cis and trans mixture) (Compound 15, 6 g, 14.1 mmol) and NaOH (3.4 g, 84.7 mmol) in EtOH/H₂O (80 mL/20 mL) was stirred at 60° C. for 2 hours. After completion (monitored by LC-MS), EtOH was removed under vacuum. The residue was acidified with HCl (1 N, aq.) to pH about 6-7. The resulting suspension was extracted with DCM (2×50 mL). The combined organic layer was washed with brine (2×25 mL), dried over anhydrous sodium sulfate and concentrated under vacuum. The residue was purified by Prep-HPLC (Method D) to give Compound 16 as racemic stereoisomer 1 and racemic stereoisomer 2.

Stereoisomer 1, second eluting isomer: ¹H NMR: (400 MHz, DMSO-d₆) δ 11.97 (br, 1H), 7.66 (d, J=8.0 Hz, 2H), 7.51 (d, J=8.0 Hz, 2H), 3.57 (d, J=14.0 Hz, 1H), 3.41 (d, J=14.0 Hz, 1H), 2.53-2.49 (m, 1H), 2.43-2.25 (m, 3H), 2.21-2.10 (m, 1H), 1.98-1.80 (m, 1H), 1.36-1.32 (m, 15H), 1.20-1.15 (m, 2H). LCMS (Method E): 398.2 (M+H), Rt. 1.757 min, 97.96% (Max). HPLC analysis (Method D): Rt. 8.41 min, 98.33% (Max). Yield: 12% (650 mg, white solid). Stereoisomer 1 of Compound 16 was arbitrarily assigned as a racemic mixture of trans enantiomers (e.g., 2-((3S,5S)-(5-cycloheptyl-1-(4-(trifluoromethyl)benzyl)piperidin-3-yl)acetic acid and 2-((3R,5R)-(5-cycloheptyl-1-(4-(trifluoromethyl)benzyl)piperidin-3-yl)acetic acid).

Stereoisomer 2, first eluting isomer: ¹H NMR (400 MHz, DMSO) δ 11.99 (br, 1H), 7.68 (d, J=8.0 Hz, 2H), 7.51 (d, J=8.0 Hz, 2H), 3.53 (dd, J=16.0, 20.0 Hz, 2H), 2.75 (t, J=6.0 Hz, 2H), 2.06 (d, J=4.0 Hz, 2H), 1.90-1.78 (m, 1H), 1.66-1.23 (m, 18H). LCMS: (Method E) 398.2 (M+H), Rt. 1.650 min. Stereisoimer 2 of Compound 16 was arbitrarily assigned as a racemic mixture of cis enantiomers (e.g., 2-((3R,5S)-5-cycloheptyl-1-(4-(trifluoromethyl)benzyl)piperidin-3-yl)acetic acid) and 2-((3S,5R)-5-cycloheptyl-1-(4-(trifluoromethyl)benzyl)piperidin-3-yl)acetic acid).

### Example 3. Chiral Separation of Enantiomers of Compound 16, Stereoisomer 1

Stereoisomer 1 of Compound 16 was arbitrarily assigned as a racemic mixture of trans enantiomers. Chiral Prep SFC (Method G) of Stereoisomer 1 of Compound 16 was performed to give a first-eluting enantiomer and a second-eluting enantiomer: Rt (First-eluting enantiomer, Enantiomer 1-A)=0.903 min; Rt (Second-eluting enantiomer, Enantiomer 1-B)=1.814.

First-eluting enantiomer (1-A): Yield: 11.0% (75.0 mg, white solid). ¹H NMR (400 MHz, DMSO-d6) δ 11.97 (br, 1H), 7.66 (d, J=8.0 Hz, 2H), 7.51 (d, J=8.0 Hz, 2H), 3.57 (d, J=14.0 Hz, 1H), 3.41 (d, J=14.0 Hz, 1H), 2.53-2.49 (m, 1H), 2.43-2.25 (m, 3H), 2.21-2.10 (m, 1H), 1.98-1.80 (m, 1H), 1.36-1.32 (m, 15H), 1.20-1.15 (m, 2H). LCMS (Method B): 398.2 (M+H), Rt. 1.77 min, 97.96% (Max). HPLC analysis (Method D): Rt. 8.41 min, 98.33% (Max). Enantiomer 1-A of Compound 16 was arbitrarily assigned as 2-((3S,5S)-5-Cycloheptyl-1-(4-(trifluoromethyl)benzyl)piperidin-3-yl)acetic acid.

Second-eluting enantiomer (1-B): ¹H NMR (400 MHz, DMSO-d6) δ 7.66 (d, J=8.0 Hz, 2H), 7.51 (d, J=8.0 Hz, 2H), 3.57 (d, J=16.0 Hz, 1H), 3.44-3.40 (m, 1H), 2.42-2.38 (m, 1H), 2.37-2.21 (m, 3H), 2.19-2.06 (m, 1H), 2.03-1.95 (m, 1H), 1.60-1.20 (m, 17H). LCMS: Method: A-0.1% NH₃·H₂O in H₂O, B-ACN; flow rate: 2.0 mL/min; column: XBridge C18 (50×4.6 mm, 3.5 μm), ESI mode; 398.4 (M+H); Rt: 1.72 min. Enantiomer 1-B of Compound 16 was arbitrarily assigned as 2-((3R,5R)-5-Cycloheptyl-1-(4-(trifluoromethyl)benzyl)piperidin-3-yl)acetic acid.

### Example 4: 2-5-cyclooctyl-1-(4-(trifluoromethyl)benzyl)piperidin-3-yl)acetic acid (Compound 21)

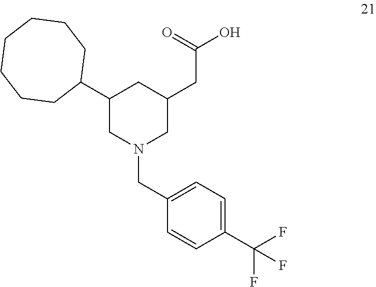 [SMILES: O=C(O)CC1CC(C2CCCCCCC2)CN(Cc2ccc(C(F)(F)F)cc2)C1]

### Step 1: (E)-ethyl 2-(5-(cyclooct-1-en-1-yl)pyridin-3-yl)acetate (Compound 17)

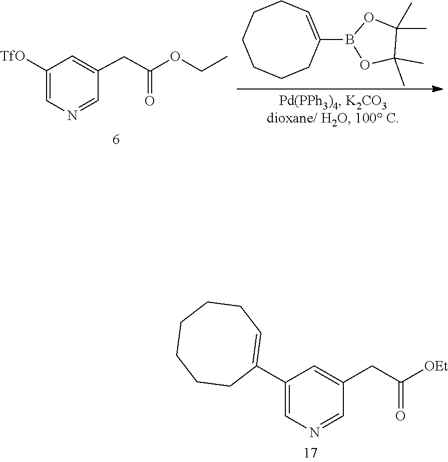

A mixture of ethyl 2-(5-(((trifluoromethyl)sulfonyl)oxy)pyridin-3-yl)acetate (Compound 6, 1.14 g, 3.64 mmol), (Z)-2-(cyclooct-1-en-1-yl)-4,4,5,5-tetramethyl-1,3,2-dioxaborolane (1.29 g, 5.47 mmol), Pd(PPh₃)₄ (84.4 mg, 0.07 mmol), K₂CO₃ (1.01 g, 7.32 mmol), dioxane (10 mL) and H₂O (2 mL) was stirred for overnight at 100° C. under N₂ atmosphere. After completion (monitored by LC-MS), organic solvent was removed under vacuum and the residue was diluted with H₂O (8 mL), extracted with EtOAc (3×20 mL). The combined organic layers were washed with brine (2×20 mL), dried over anhydrous sodium sulfate, filtered, and concentrated. The residue was purified by flash chromatography on silica gel (PE/EA=20:1-5:1) to give (E)-ethyl 2-(5-(cyclooct-1-en-1-yl)pyridin-3-yl)acetate (Compound 17). Yield: 66% (655.8 mg).

### Step 2: (E)-3-(cyclooct-1-en-1-yl)-5-(2-ethoxy-2-oxoethyl)-1-(4-(trifluoromethyl)benzyl)pyridin-1-ium bromide (Compound 18)

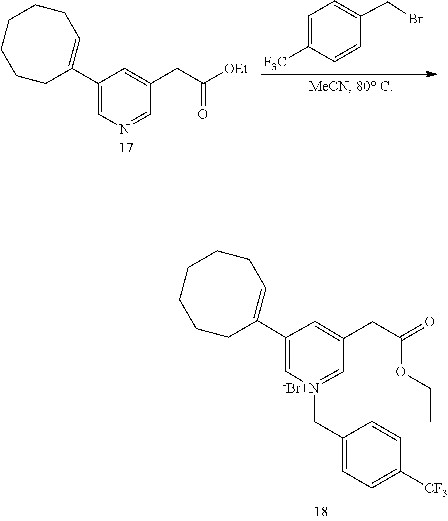

A solution of (E)-ethyl 2-(5-(cyclooct-1-en-1-yl)pyridin-3-yl)acetate (Compound 17, 655.8 mg, 2.4 mmol) and 1-(bromomethyl)-4-(trifluoromethyl)benzene (688.1 mg, 2.9 mmol) in MeCN (10 mL) was stirred at 80° C. for 7 hours. After completion (monitored by TLC), the reaction mixture was concentrated under vacuum to give (E)-3-(cyclooct-1-en-1-yl)-5-(2-ethoxy-2-oxoethyl)-1-(4-(trifluoromethyl)benzyl)pyridin-1-ium bromide (Compound 18). Yield: crude (1.34 g, yellow oil).

### Step 3: (E)-ethyl 2-(5-(cyclooct-1-en-1-yl)-1-(4-(trifluoromethyl)benzyl)-1,2,3,4-tetrahydropyridin-3-yl)acetate (Compound 19)

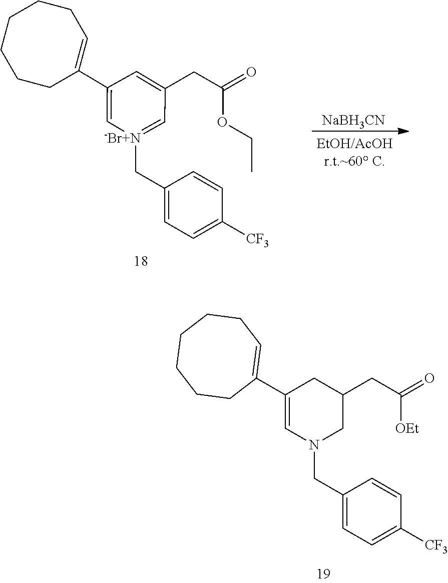

To a solution of (E)-3-(cyclooct-1-en-1-yl)-5-(2-ethoxy-2-oxoethyl)-1-(4-(trifluoromethyl)benzyl)pyridin-1-ium bromide (Compound 18, 1.3 g, crude) in EtOH/AcOH (10 mL/1 mL) was added NaBH₃CN (1.63 g, 25.9 mmol) in batches at room temperature. The reaction mixture was stirred for overnight at 60° C. After completion (monitored by LC-MS), the reaction mixture was quenched with water (15 mL). The mixture was extracted with EA (3×30 mL). The combined organic layers were washed with brine (2×25 mL), dried over anhydrous sodium sulfate, filtered, and concentrated to get (E)-ethyl 2-(5-(cyclooct-1-en-1-yl)-1-(4-(trifluoromethyl)benzyl)-1,2,3,4-tetrahydropyridin-3-yl)acetate (Compound 19). Yield: crude (1.07 g). LCMS: (Method E) 436.4 (M+H).

### Step 4: ethyl 2-(5-cyclooctyl-1-(4-(trifluoromethyl)benzyl)piperidin-3-yl)acetate (Compound 20)

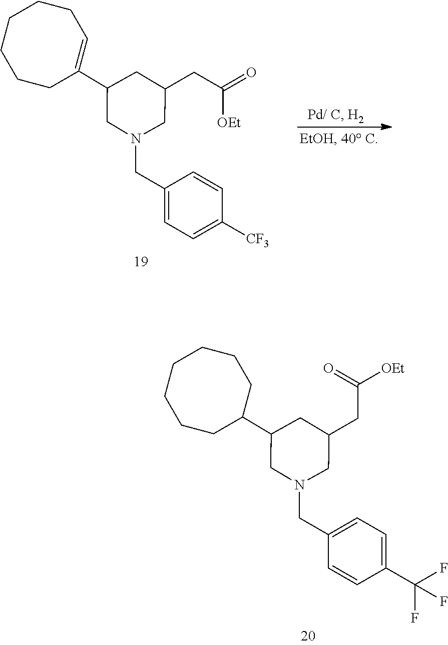 [SMILES: CCOC(=O)CC1CC(/C2=C/CCCCCC2)CN(Cc2ccc(C(F)(F)F)cc2)C1 ; "Pd/ C, H2" ; CCOC(=O)CC1CC(C2CCCCCCC2)CN(Cc2ccc(C(F)(F)F)cc2)C1]

A mixture of (E)-ethyl 2-(5-(cyclooct-1-en-1-yl)-1-(4-(trifluoromethyl)benzyl)-1,2,3,4-tetrahydropyridin-3-yl)acetate (Compound 19, 1.07 g, crude) and Pd/C (10 wt %, 784.2 mg) in EtOH (10 mL) was stirred for overnight at 40° C. under H₂. After completion (monitored by LCMS), the reaction mixture was filtered, and the filtrate was concentrated under vacuum. The residue was purified by flash chromatography on silica gel (PE/EA=20:1-5:1) to give a first-eluting isomer and a second-eluting isomer of 2-(5-cyclooctyl-1-(4-(trifluoromethyl)benzyl)piperidin-3-yl)acetate (Compound 20).

First-eluting isomer, silica gel chromatography: Yield: 9.3% (98.7 mg). LCMS: (Method E) 440.4 (M+H), Rt. 2.801 min. Stereoisomer 1 of Compound 20 was arbitrarily assigned as a racemic mixture of trans enantiomers (e.g., ethyl 2-((3S,5S)-5-cyclooctyl-1-(4-(trifluoromethyl)benzyl)piperidin-3-yl)acetate and ethyl 2-((3R,5R)-5-cyclooctyl-1-(4-(trifluoromethyl)benzyl)piperidin-3-yl)acetate).

Second-eluting isomer, silica gel chromatography: Yield: 11.7% (125.1 mg). LCMS: (Method E) 440.4 (M+H), Rt. 2.647 min. Stereoisomer 2 of Compound 20 was arbitrarily assigned as a racemic mixture of cis enantiomers (e.g., ethyl 2-((3R,5S)-5-cyclooctyl-1-(4-(trifluoromethyl)benzyl)piperidin-3-yl)acetate and ethyl 2-((3S,5R)-5-cyclooctyl-1-(4-(trifluoromethyl)benzyl)piperidin-3-yl)acetate).

### Step 5: 2-(5-cyclooctyl-1-(4-(trifluoromethyl)benzyl)piperidin-3-yl)acetic acid (Compound 21)

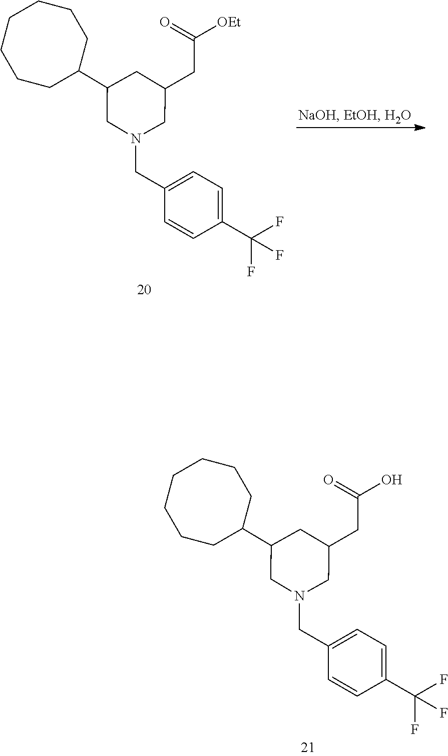

A solution of the first-eluting isomer of Compound 20 (98.7 mg, 0.22 mmol) and NaOH (53.9 mg, 1.35 mmol) in EtOH/H₂O (2.4 mL/0.6 mL) was stirred at 60° C. for 2 hours. After completion (monitored by LC-MS), EtOH was removed under vacuum. The residue was acidified with HCl (1 N, aq.) to pH about 5-6. The resulting suspension was extracted with EA (3×10 mL). The combined organic layer was washed with brine (2×10 mL), dried over anhydrous sodium sulfate and concentrated under vacuum. The residue was purified by Prep-HPLC (Method C) to give Compound 21 as Stereoisomer 1. Yield: 52% (47.6 mg). ¹H NMR (400 MHz, DMSO-d₆) δ 11.92 (br, 1H), 7.66 (d, J=8.0 Hz, 2H), 7.51 (d, J=8.0 Hz, 2H), 3.57 (d, J=14.0 Hz, 1H), 3.41 (d, J=14.0 Hz, 1H), 2.44-2.31 (m, 1H), 2.31-2.17 (m, 3H), 2.17-1.96 (m, 2H), 1.74-1.04 (m, 19H). LCMS: (Method E) 412.3 (M+H), Rt. 1.79 min, 100.0% (Max). HPLC analysis (Method D) Rt. 8.64 min, 100.0% (Max). Stereoisomer 1 of Compound 21 was arbitrarily assigned as a racemic mixture of trans enantiomers (e.g., 2-((3S,5S)-5-cyclooctyl-1-(4-(trifluoromethyl)benzyl)piperidin-3-yl)acetic acid and 2-((3R,5R)-5-cyclooctyl-1-(4-(trifluoromethyl)benzyl)piperidin-3-yl)acetic acid).

A solution of the second-eluting isomer of Compound 20 (125.1 mg, 0.28 mmol) and NaOH (68.3 mg, 1.7 mmol) in EtOH/H₂O (2.4 mL/0.6 mL) was stirred at 60° C. for 2 hours. After completion (monitored by LC-MS), EtOH was removed under vacuum. The residue was acidified with HCl (1 N, aq.) to pH about 5-6. The resulting suspension was extracted with EA (3×10 mL). The combined organic layer was washed with brine (2×10 mL), dried over anhydrous sodium sulfate and concentrated under vacuum. The residue was purified by Prep-HPLC (Method C) to give Compound 21 as stereoisomer 2. Yield: 48% (56.4 mg). ¹H NMR (400 MHz, DMSO-d₆) δ 12.02 (br, 1H), 7.67 (d, J=8.0 Hz, 2H), 7.51 (d, J=8.0 Hz, 2H), 3.59-3.49 (m, 2H), 2.78 (t, J=7.6 Hz, 2H), 2.06 (d, J=7.2 Hz, 2H), 1.99-1.80 (m, 1H), 1.76-1.70 (m, 1H), 1.67-1.17 (m, 18H), 0.71-0.62 (m, 1H). LCMS: (Method E) 412.4 (M+H), Rt. 1.66 min, 100.0% (Max). HPLC analysis (Method D) Rt. 7.69 min, 100.0% (Max). Stereoisomer 2 of Compound 21 was arbitrarily assigned as a racemic mixture of cis enantiomers (e.g., 2-((3R,5S)-5-cyclooctyl-1-(4-(trifluoromethyl)benzyl)piperidin-3-yl)acetic acid and 2-((3S,5R)-5-cyclooctyl-1-(4-(trifluoromethyl)benzyl)piperidin-3-yl)acetic acid)).

### Example 5: Synthesis of 2-1-(4-(trifluoromethyl)benzyl)-5-((E)-4-(trifluoromethyl)styryl)piperidin-3-yl)acetic acid (Compound 25)

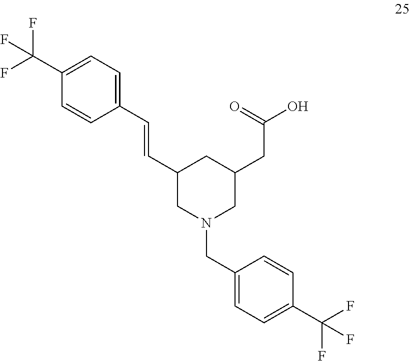 [SMILES: O=C(O)CC1CC(/C=C/c2ccc(C(F)(F)F)cc2)CN(Cc2ccc(C(F)(F)F)cc2)C1]

### Step 1: (E)-ethyl 2-(5-(4-(trifluoromethyl)styryl)pyridin-3-yl)acetate (Compound 22)

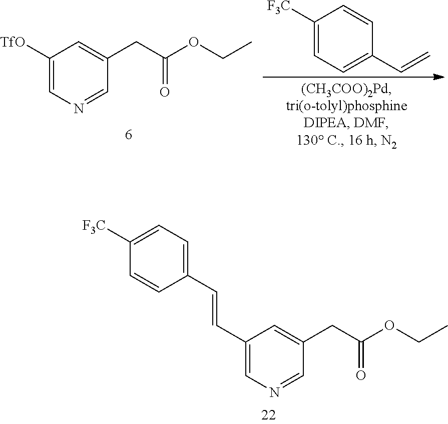 [SMILES: CCOC(=O)Cc1cncc(OS(=O)(=O)C(F)(F)F)c1 ; C=Cc1ccc(C(F)(F)F)cc1 ; CCOC(=O)Cc1cncc(/C=C/c2ccc(C(F)(F)F)cc2)c1]

To a solution of ethyl 2-(5-(((trifluoromethyl)sulfonyl)oxy) pyridin-3-yl)acetate (Compound 6, 1.0 g, 3.19 mmol), 1-(trifluoromethyl)-4-vinylbenzene (560.0 mg, 3.19 mmol) and Palladium (II) acetate (72 mg, 0.32 mmol) in DMF (15 mL) was added tri(o-tolyl)phosphine (196.0 mg, 0.64 mmol) and N,N-diisopropylethylamine (DIPEA, 1.2 g, 9.57 mmol). The reaction mixture was stirred at 130° C. under N₂ for 16 h. After completion (monitored by TLC and LC-MS), the reaction mixture was poured into water. The mixture was extracted with EtOAc (3×50 mL). The combined organic layers were washed with brine (3×20 mL), dried over anhydrous sodium sulfate, filtered, and concentrated. The residue was further purified by flash chromatography on silica gel (PE/EA=20:1-4:1) to give (E)-ethyl 2-(5-(4-(trifluoromethyl)styryl)pyridin-3-yl)acetate (Compound 22). Yield: crude (560 mg, yellow oil). LCMS: (Method F) 336.3 (M+H). ¹H NMR (400 MHz, CDCl₃) δ 8.65 (d, J=1.3 Hz, 1H), 8.44 (s, 1H), 7.82 (s, 1H), 7.73-7.52 (m, 4H), 7.20 (d, J=16.4 Hz, 1H), 7.14 (d, J=16.5 Hz, 1H), 4.20 (q, J=7.1 Hz, 2H), 3.66 (s, 2H), 1.28 (t, J=7.1 Hz, 3H).

### Step 2: (E)-3-(2-ethoxy-2-oxoethyl)-1-(4-(trifluoromethyl)benzyl)-5-(4-(trifluoromethyl)styryl)pyridin-1-ium bromide (Compound 23)

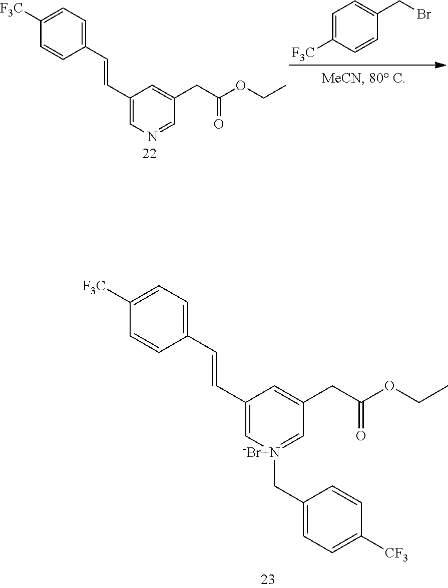

A solution of ethyl (E)-2-(5-(4-(trifluoromethyl)styryl)pyridin-3-yl)acetate (Compound 22, 560 mg, crude) and 1-(bromomethyl)-4-(trifluoromethyl) benzene (400 mg, 1.7 mmol) in MeCN (20 mL) was stirred at 80° C. for 7 hours. After completion (monitored by TLC), the reaction mixture was concentrated under vacuum and the residue was purified by flash chromatography on silica gel (DCM/MeOH=10:1-1:1) to give (E)-3-(2-ethoxy-2-oxoethyl)-1-(4-(trifluoromethyl)benzyl)-5-(4-(trifluoromethyl)styryl)pyridin-1-ium bromide (Compound 23). Yield: 60.4% (590 mg, white solid).

### Step 3: (E)-ethyl 2-(1-(4-(trifluoromethyl)benzyl)-5-(4-(trifluoromethyl)styryl)piperidin-3-yl)acetate (Compound 24)

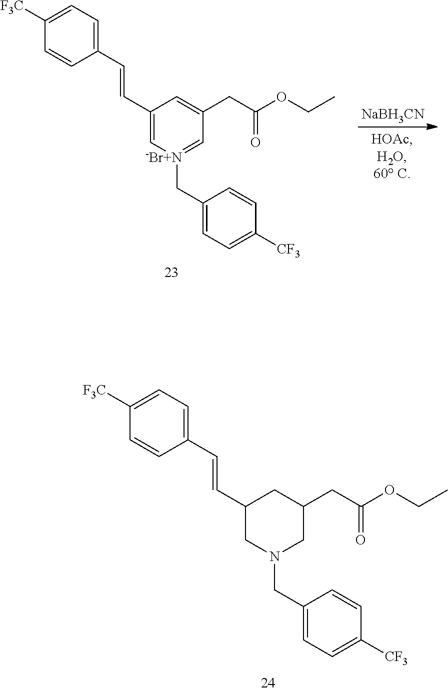

To a solution of (E)-3-(2-ethoxy-2-oxoethyl)-1-(4-(trifluoromethyl)benzyl)-5-(4-(trifluoromethyl)styryl)pyridin-1-ium bromide (Compound 23, 590.0 mg, 1.03 mmol) in EtOH/AcOH (20 mL/2 mL) was added NaBH₃CN (1.29 g, 20.54 mmol) in batches at room temperature. The reaction mixture was stirred at 60° C. After completion (monitored by LC-MS), the reaction mixture was neutralized with 10% sodium carbonate aqueous solution. The mixture was extracted with DCM (3×30 mL). The combined organic layers were washed with brine (2×20 mL), dried over anhydrous sodium sulfate, filtered, and concentrated to give (E)-ethyl 2-(1-(4-(trifluoromethyl)benzyl)-5-(4-(trifluoromethyl)styryl)piperidin-3-yl)acetate (Compound 24). Yield: crude (120 mg, yellow oil). LCMS: (Method F) 500.5 (M+H).

### Step 4: (E)-2-(1-(4-(trifluoromethyl)benzyl)-5-(4-(trifluoromethyl)styryl)piperidin-3-yl)acetic acid (Compound 25)

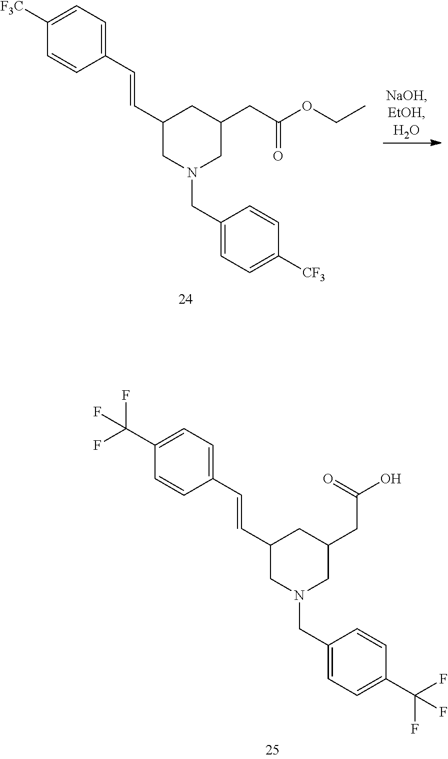 [SMILES: CCOC(=O)CC1CC(/C=C/c2ccc(C(F)(F)F)cc2)CN(Cc2ccc(C(F)(F)F)cc2)C1 ; O=C(O)CC1CC(/C=C/c2ccc(C(F)(F)F)cc2)CN(Cc2ccc(C(F)(F)F)cc2)C1]

A solution of ethyl (E)-2-(1-(4-(trifluoromethyl)benzyl)-5-(4-(tri-fluoromethyl)styryl)piperidin-3-yl)acetate (Compound 23, 120 mg, crude) and NaOH (97.0 mg, 2.4 mmol) in EtOH/H₂O (2 mL/0.5 mL) was stirred at 60° C. for 2 hours. After completion (monitored by LC-MS), EtOH was removed under vacuum. The residue was acidified with HCl (1N, aq.) to pH about 6-7. The resulting suspension was extracted with DCM (2×5 mL). The combined organic layer was washed with brine (2×2 mL), dried over anhydrous sodium sulfate and concentrated under vacuum. The residue was further purified by Prep-HPLC (Method D) to give Compound 25 as a first-eluting isomer and a second-eluting isomer.

First-eluting isomer HPLC: HPLC analysis (Method E) Rt. 7.35 min. Arbitrarily assigned as a racemic mixture of cis enantiomers (e.g., 2-((3R,5S)-1-(4-(trifluoromethyl)benzyl)-5-((E)-4-(trifluoromethyl)styryl)piperidin-3-yl)acetic acid and 2-((3S,5R)-1-(4-(trifluoromethyl)benzyl)-5-((E)-4-(trifluoromethyl)styryl)piperidin-3-yl)acetic acid).

Second-eluting isomer HPLC: Yield: 3.5% (4 mg, white solid). ¹H NMR (400 MHz, DMSO-d₆) δ 12.02 (br, 1H), 7.68 (dd, J=19.2, 10.8 Hz, 4H), 7.60 (d, J=8.0 Hz, 2H), 7.55 (d, J=8.0 Hz, 2H), 6.53 (s, 2H), 3.56 (dd, J=31.2, 14.0 Hz, 2H), 2.67-2.57 (m, 2H), 2.34-2.24 (m, 6H), 1.56-1.54 (m, 2H). LCMS: (Method F) 472.2 (M+H), Rt. 1.46 min, 100.00% (Max). HPLC analysis (Method E) Rt. 7.78 min, 96.87% (Max). Arbitrarily assigned as a racemic mixture of trans enantiomers (e.g., 2-((3S,5S)-1-(4-(trifluoromethyl)benzyl)-5-((E)-4-(trifluoromethyl)styryl)piperidin-3-yl)acetic acid and 2-((3R,5R)-1-(4-(trifluoromethyl)benzyl)-5-((E)-4-(trifluoromethyl)styryl)piperidin-3-yl)acetic acid).

### Example 6: Synthesis of 2-(1-(4-(trifluoromethyl)benzyl)-5-(4-(trifluoromethyl)phenyl)piperidin-3-yl)cyclopropane-1-carboxylic acid (Compound 31)

2-(1-(4-(trifluoromethyl)benzyl)-5-(4-(trifluoromethyl)phenyl)piperidin-3-yl)cyclopropane-1-carboxylic acid (Compound 31) was prepared according to the scheme below.

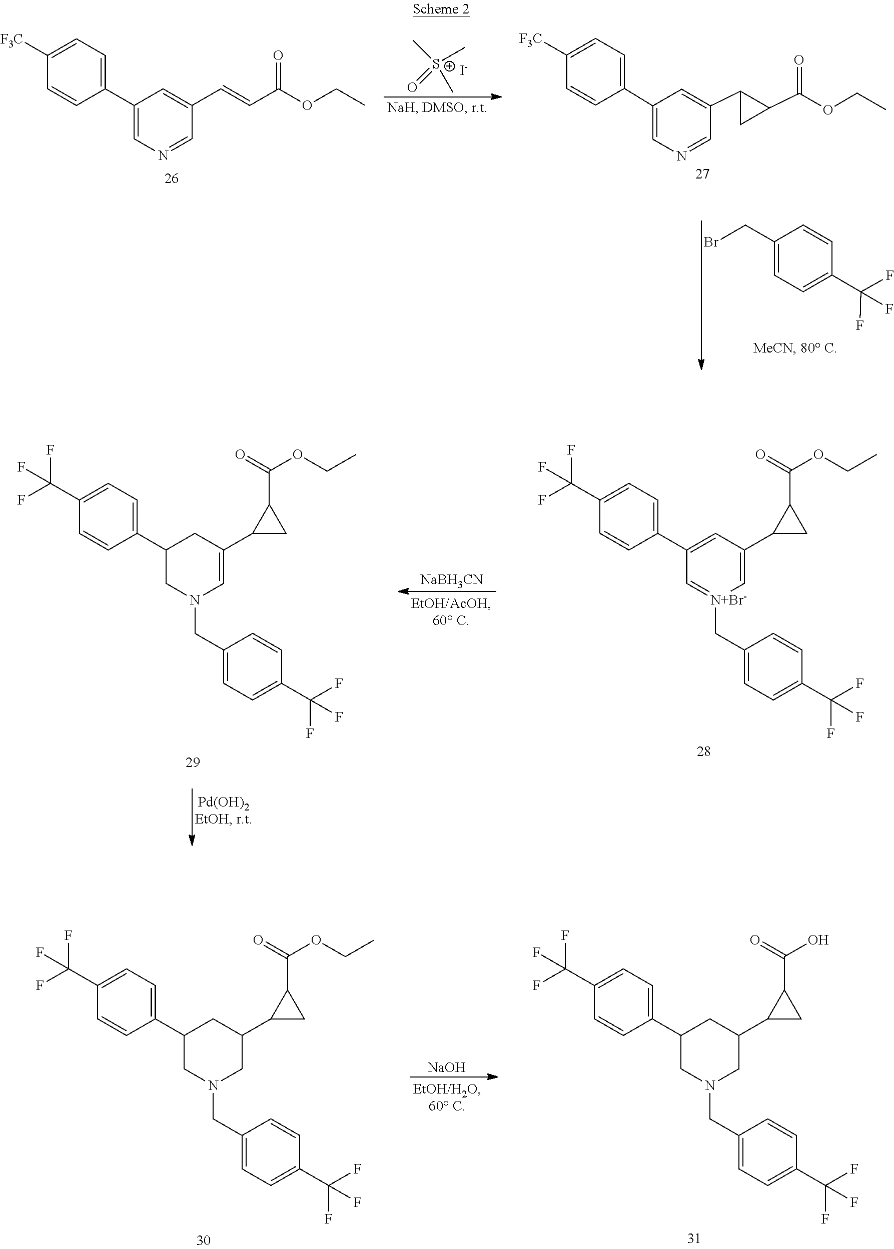

### Step 1: ethyl 2-(5-(4-(trifluoromethyl)phenyl)pyridin-3-yl)cyclo-propane-1-carboxylate (Compound 27)

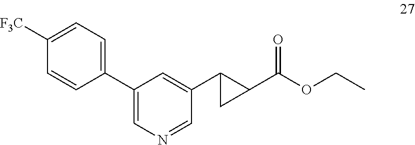 [SMILES: CCOC(=O)C1CC1c1cncc(-c2ccc(C(F)(F)F)cc2)c1]

To a suspension of NaH (51.0 mg, 1.27 mmol) in dry dimethyl sulfoxide (DMSO, 1 mL) was added trimethylsulfoxonium iodide (280.0 mg, 1.27 mmol). The reaction mixture was stirred at room temperature under N₂ for 1 h until a clear solution was formed. Then, a solution of ethyl (E)-3-(5-(4-(trifluoromethyl)phenyl)pyridin-3-yl)acrylate (Compound 26, 340.2 mg, 1.06 mmol) in dry DMSO (2 mL) was added dropwise and stirred at room temperature for 3 hours. After completion (monitored by TLC and LC-MS), the reaction mixture was poured into ice water. The mixture was extracted with EtOAc (3×50 mL). The combined organic layers were washed with brine (2×20 mL), dried over anhydrous sodium sulfate, filtered, and concentrated. The residue was further purified by flash chromatography on silica gel to give ethyl 2-(5-(4-(trifluoromethyl)phenyl)pyridin-3-yl)cyclo-propane-1-carboxylate (Compound 27). Yield: crude (170 mg, white solid). LCMS: (Method F) 336.3 (M+H).

### Step 2: 3-(2-(ethoxycarbonyl)cyclopropyl)-1-(4-(trifluoromethyl)benzyl)-5-(4-(tri-fluoromethyl)phen-yl)py-ridin-1-ium bromide (Compound 28)

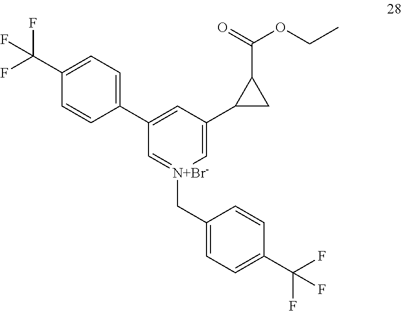

A solution of ethyl 2-(5-(4-(trifluoromethyl)phenyl)pyridin-3-yl) cyclopropane-1-carboxylate (Compound 27, 170 mg, crude) and 1-(bromomethyl)-4-(trifluoromethyl) benzene (122 mg, 0.51 mmol) in MeCN (5 mL) was stirred at 80° C. for 7 hours. After completion (monitored by TLC), the reaction mixture was concentrated under vacuum and the residue was purified by flash chromatography on silica gel to give 3-(2-(ethoxycarbonyl)cyclopropyl)-1-(4-(trifluoromethyl)benzyl)-5-(4-(tri-fluoromethyl)phen-yl)py-ridin-1-ium bromide (Compound 28). Yield: 65% (190 mg, white solid).

### Step 3: ethyl 2-(1-(4-(trifluoromethyl)benzyl)-5-(4-(trifluoromethyl)phenyl)-1,4,5,6-tetrahydropyridin-3-yl)cyclopropane-1-carboxylate (Compound 29)

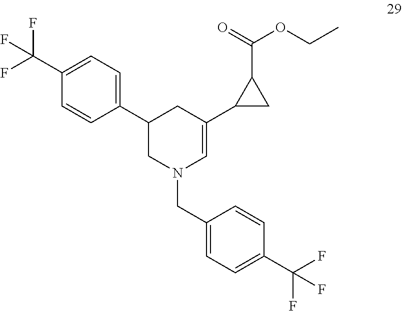 [SMILES: CCOC(=O)C1CC1C1=CN(Cc2ccc(C(F)(F)F)cc2)CC(c2ccc(C(F)(F)F)cc2)C1]

To a solution of 3-(2-(ethoxycarbonyl)cyclopropyl)-1-(4-(trifluoromethyl) benzyl)-5-(4-(trifluoromethyl)phen-yl)pyridin-1-ium bromide (Compound 28, 190.1 mg, 0.33 mmol) in EtOH/AcOH (10 mL/1 mL) was added NaBH₃CN (415.8 mg, 6.6 mmol) in batches at room temperature. The reaction mixture was stirred at 60° C. After completion (monitored by LC-MS), the reaction mixture was neutralized with 10% Na₂CO₃ aqueous solution. The mixture was extracted with DCM (3×30 mL). The combined organic layers were washed with brine (2×20 mL), dried over anhydrous sodium sulfate, filtered, and concentrated to get the crude product. Yield: crude (200 mg, yellow oil). LCMS: (Method F) 498.5 (M+H).

### Step 4: ethyl 2-(1-(4-(trifluoromethyl)benzyl)-5-(4-(trifluoromethyl)phenyl)piperidin-3-yl)cyclopropane-1-carboxylate (Compound 30)

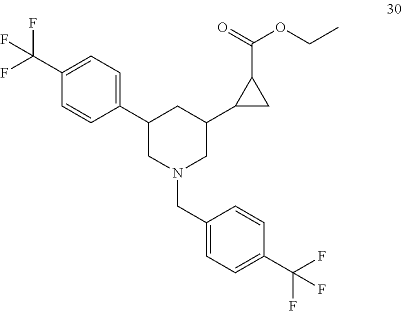 [SMILES: CCOC(=O)C1CC1C1CC(c2ccc(C(F)(F)F)cc2)CN(Cc2ccc(C(F)(F)F)cc2)C1]

A mixture of ethyl 2-(1-(4-(trifluoromethyl) benzyl)-5-(4-(trifluoro-methyl)phenyl)-1,4,5,6-tetrahydropyridin-3-yl) cyclopropane-1-carboxylate (Compound 29, crude, 200.0 mg, 0.40 mmol) and Pd/C (10 wt %, 200 mg) in EtOH (10 mL) was stirred under H₂ at 40° C. After completion (monitored by LC-MS), the reaction mixture was filtered, and the filtrate was concentrated under vacuum. The residue was purified by prep-HPLC purification (Method E) to give a first-eluting isomer and a second-eluting isomer of ethyl 2-(1-(4-(trifluoromethyl)benzyl)-5-(4-(trifluoromethyl)phenyl)piperidin-3-yl)cyclopropane-1-carboxylate (Compound 30).

First-eluting isomer HPLC: LCMS: (Method F) Rt=1.599 min.

Second-eluting isomer HPLC: Yield: 5.1% (10.0 mg, white solid). LCMS: (Method F) 500.2 (M+H), Rt. 1.744 min.

### Step 5: 2(-1-(4-(trifluoromethyl)benzyl)-5-(4-(trifluoromethyl)phenyl)piperidin-3-yl)cyclopropane-1-carboxylic acid (Compound 31)

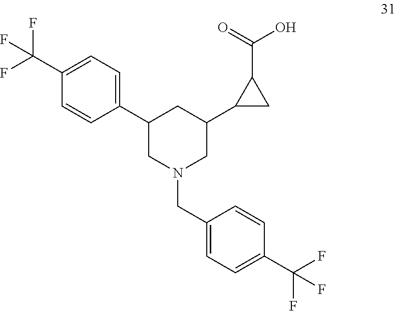 [SMILES: O=C(O)C1CC1C1CC(c2ccc(C(F)(F)F)cc2)CN(Cc2ccc(C(F)(F)F)cc2)C1]

A solution of the second-eluting isomer of ethyl 2-(1-(4-(trifluoromethyl)benzyl)-5-(4-(trifluoromethyl) phenyl)piperidin-3-yl)cyclopropane-1-carboxylate (Compound 30, 10 mg, 0.02 mmol) and NaOH (8 mg, 0.2 mmol) in EtOH/H₂O (0.4 mL/0.1 mL) was stirred at 60° C. for 2 hours. After completion (monitored by LC-MS), EtOH was removed under vacuum. The residue was acidified with aq. HCl (1 N) to pH about 6-7. The resulting suspension was extracted with DCM (2×1 mL). The combined organic layer was washed with brine (2×2 mL), dried over anhydrous sodium sulfate and concentrated under vacuum. The residue was further purified by prep-HPLC purification (Method D) to give 2-(1-(4-(trifluoromethyl)benzyl)-5-(4-(trifluoromethyl)phenyl)piperidin-3-yl)cyclopropane-1-carboxylic acid (Compound 31) as Stereoisomer 1. Yield: 7.4% (0.7 mg, white solid). ¹H NMR (400 MHz, CD₃OD) δ 7.63-7.49 (m, 8H), 3.80-3.46 (m, 2H), 3.24 (m, 1H), 2.92-2.86 (m, 1H), 2.76-2.64 (m, 1H), 2.45-2.30 (m, 2H), 2.08-1.84 (m, 3H), 1.32-1.28 (m, 1H), 1.12-1.07 (m, 2H), 0.77-0.62 (m, 1H). LCMS: (Method F) 472.2 (M+H), Rt. 1.46 min, 100.00% (Max). HPLC: (Method D) Rt. 7.68 min, 100.0% (Max). Stereoisomer 1 of Compound 31 was arbitrarily assigned as a racemic mixture of (2R)-2-((3S,5S)-1-(4-(trifluoromethyl)benzyl)-5-(4-(trifluoromethyl)phenyl)piperidin-3-yl)cyclopropane-1-carboxylic acid and (2S)-2-((3R,5R)-1-(4-(trifluoromethyl)benzyl)-5-(4-(trifluoromethyl)phenyl)piperidin-3-yl)cyclopropane-1-carboxylic acid.

The above step was conducted with the first-eluting isomer of Compound 30 to give Compound 31 as stereoisomer 2. ¹H NMR (400 MHz, CD₃OD) δ 7.65 (d, J=8.0 Hz, 2H), 7.61-7.52 (m, 6H), 3.68 (dd, J=16.0, 24.0 Hz, 2H), 3.27-3.24 (m, 1H), 2.89-2.85 (m, 2H), 2.62-2.54 (m, 3H), 2.20-2.13 (m, 1H), 1.94-1.85 (m, 1H). LCMS (Method F) 472.2 (M+H), Rt. 1.39 min. Stereoisomer 2 was assigned as a racemic mixture of (2R)-2-((3S,5R)-1-(4-(trifluoromethyl)benzyl)-5-(4-(trifluoromethyl)phenyl)piperidin-3-yl)cyclopropane-1-carboxylic acid and (2S)-2-((3R,5S)-1-(4-(trifluoromethyl)benzyl)-5-(4-(trifluoromethyl)phenyl)piperidin-3-yl)cyclopropane-1-carboxylic acid.

### Example 7. Synthesis of Other Compounds

The following compounds shown in Table 1 were synthesized using similar methods to the proceeding compounds. Mass spectrometry (MS), ¹H NMR, and LCMS characterization data is included for each compound

TABLE 1

| | | MS | | LCMS |
|---|---|---|---|---|
| Compound structure | Isomer/Compound name | characterization | ¹H NMR characterization | characterization |
| 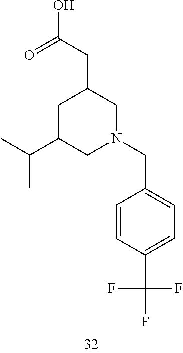 [SMILES: CC(C)C1CC(CC(=O)O)CN(Cc2ccc(C(F)(F)F)cc2)C1] | Stereoisomer 1: Arbitrarily assigned as a racemic misxture of 2-((3R,5S)-5-isopropyl-1-(4- (trifluoromethyl)benzyl)piperidin- 3-yl)acetic acid and 2-((3S,5R)-5- isopropyl-1-(4- (trifluoromethyl)benzyl)piperidin- 3-yl)acetic acid | MS: 344.2 (M + H) | (400 MHz, DMSO) δ 12.08 (br, 1H), 7.67 (d, J = 8.0 Hz, 2H), 7.51 (d, J = 8.0 Hz, 2H), 3.55 (dd, J = 14.0, 32.8 Hz, 2H), 2.79-2.73 (m, 2H), 2.07 (d, J = 6.8 Hz, 2H), 1.90-1.77 (m, 1H), 1.76-1.74 (m, 1H), 1.61-1.51 (m, 2H), 1.39- 1.34 (m, 2H), 0.84-0.80 (m, 6H), 0.56 (dd, J = 24.0, 48.0 Hz, 1H). | Method: A-0.1% NH₃•H₂0 in H2O, B-ACN; flow rate: 2.0 mL/min; column: XBridge C18 (50 × 4.6 mm, 3.5 μm), ESI mode. Rt: 1.23 min |
| | Stereoisomer 2: Arbitrarily | MS: 344.3 | (400 MHz, DMSO) δ 12.12 | Method: A-0.1% |
| | assigned as a racemic mixture of | (M + H) | (br, 1H), 7.66 (d, J = 8.0 Hz, | NH₃•H₂0 in |
| | 2-((3R,5R)-5-isopropyl-1-(4- | | 2H), 7.51 (d, J = 8.0 Hz, | H2O, B-ACN; |
| | (trifluoromethyl)benzyl)piperidin- | | 2H), 3.59 (d, J = 14.0 Hz, | flow rate: |
| | 3-yl)acetic acid and 2-((3S,5S)-5- | | 1H), 3.41 (d, J = 14.4 Hz, | 2.0 mL/min; |
| | isopropyl-1-(4- | | 1H), 2.51-2.50 (m, 1H), 2.49- | column: |
| | (trifluoromethyl)benzyl)piperidin- | | 2.40 (m, 1H), 2.38-2.26 (m, | XBridge C18 |
| | 3-yl)acetic acid | | 3H), 2.26-1.99 (m, 2H), 1.57- | (50 × 4.6 mm, |
| | | | 1.53 (m, 1H), 1.37-1.34 (m, | 3.5 μm), |
| | | | 3H), 0.83 (d, J = 6.8 Hz, | ESI mode |
| | | | 3H), 0.78 (d, J = 6.4 Hz, 3H). | Rt: 1.26 min |

### Example 8. Assay for XPO1 Inhibition

HepG2 cancer cell lines were seeded at 250,000 cells per well in 6 well plates and grown for 24 hrs in the presence of DMSO control, 0.2 uM, or 1 uM of each test compound. After 24 hrs, protein lysates were harvested and XPO1 protein levels were measured by western blot. HSP70 was used as an internal loading control.

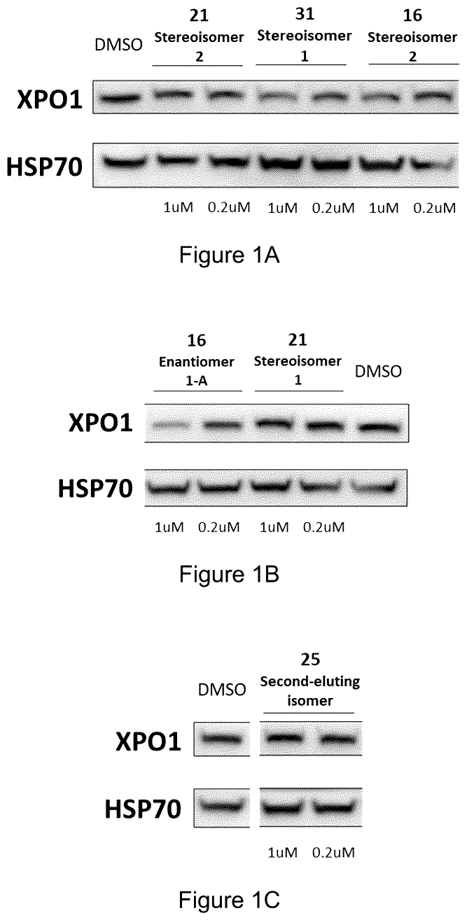

FIG. 1A shows the western blot for Compound 21, stereisomer 2; Compound 31, stereisomer 1; Compound 16, stereoisomer 2; and DMSO control. FIG. 1B shows the western blot for Compound 16, enantiomer 1-A; Compound 21, stereoisomer 1; and DMSO control. FIG. 1C shows the western blot for Compound 25, second-eluting isomer, and DMSO control. Of the tested compounds, Compound 16, enantiomer 1-A, demonstrated the largest reduction of XPO1 protein levels as can be seen by the reduced XPO1 bands at both 0.2 uM and 1 uM. Compound 16, stereoisomer 2, and Compound 31, stereoisomer 1, showed reduction of XPO1 protein at 1 uM with minor reductions observed at 0.2 uM. Finally, Compound 21, stereoisomer 1; Compound 21, stereoisomer 2; and Compound 25, second-eluting isomer, did not show evidence of a dose-dependent reduction of XPO1 protein.

### Example 9. Activity Transport Assay

Hela cells were grown in a T75 flask with complete 10% FBS DMEM medium. When the Hela cells reached over 90% confluence, the medium was replaced with FBS free Opti-MEM medium. The T75 flask of cell was transfected with 20 ug of the EYFP₂—SV40^{NLS}-REV(HIV)^{NES} reporter plasmid (Fu et. al. Molec Biol Cell 29, 2037-2044 (2018)), by using Lipofectamine 3000 following the manufacturer's instructions. After 12 hours, the medium was replaced with complete 10% FBS medium. 24 hours post-transfection, the Hela cells were trypsenized and seeded into 96-well plates at 3×10⁴ cells per well density.

36 Hours post transfection, the Hela cells were treated with Example compounds at 5 uM, 1.5 uM, and 0.5 uM concentrations. 9 wells were treated with DMSO as a negative control, and three wells were treated with 5 nM LMB as a positive control. After 18 hours of drug treatment, the cells were washed three times with PBS, and then fixed with PBS containing 4% paraformaldehyde at room temperature for 20 minutes. The cells were the treated with PBS containing 0.1% Triton-X 100 for 5 minutes at room temperature to increase permeability, then cells were washed with ice cold PBS three times. To detect the cell bodies, 100 nM TRITC labeled phalloidin was incubated with the cells for 60 minutes at 4 degrees Celsius. Then the cells were washed three times with ice cold PBS. Cells were then incubated with 5 ug/mL Hoechst 33342 for 20 minutes at room temperature to label the nucleus, followed by washing three times with ice cold PBS. Z-stack (number of planes=7, Distance=1.5 um) images were acquired by Operetta (each well acquired 25 fields of images).

The analysis was performed by Harmoony Sofware. The maximum intensity projection images were created for analysis. The Hoechst channel was used to identify the nuclear region, and the TRITC labeled phalloidin channel was used to identify the cell body. The EYFP mean fluorescence intensity over 1000 cells were selected to be successfully transfected cells. In these cells, the ratio of cytoplasmic to nuclear mean fluorescence EFYP (R_{C/N}) was calculated.

IC₅₀ data for the Example compounds is provided below in Table 2.

TABLE 2

| Compound | IC₅₀ |
|---|---|
| 11, Stereoisomer 1 | + |
| 16, Stereoisomer 1 | IA |
| 16, Stereoisomer 2 | + |
| 16, Enantiomer 1-A | +++ |
| 16, Enantiomer 1-B | IA |
| 21, Stereoisomer 1 | + |
| 21, Stereoisomer 2 | IA |
| 25, Second-eluting isomer | +++ |
| 31, Stereoisomer 1 | + |
| 31, Stereoisomer 2 | IA |
| 32, Stereoisomer 1 | IA |
| 32, Stereoisomer 2 | + |

IA refers to >5 μM
+ refers to IC₅₀ of ≤5 μM to >1.5 μM
++ refers to IC₅₀ of ≤1.5 μM to >0.5 μM
+++ refers to IC₅₀ of <0.5 μM

1. A compound of Formula I: 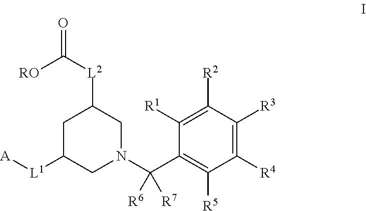
or a pharmaceutically acceptable salt thereof, wherein:
A is C₃₋₁₀ alkyl, C₃₋₁₀ cycloalkyl, 3-14 membered heterocycloalkyl, or phenyl, each optionally substituted with 1, 2, or 3 R^{A};
L¹ is absent, C₁₋₈ alkylene, C₃₋₈ cycloalkylene, or C₂₋₈ alkenylene, wherein the C₁₋₈ alkylene, C₃₋₈ cycloalkylene, or C₂₋₈ alkenylene is optionally substituted by 1, 2, or 3 substituents independently selected from C₁₋₄ alkyl, C₁₋₄haloalkyl, C₁₋₄ alkoxy, F, Cl, Br, and OH;
L² is methylene, C₂₋₈ alkenylene, or C₃₋₈ cycloalkylene, each optionally substituted by 1, 2, or 3 substituents independently selected from C₁₋₄ alkyl, C₁₋₄haloalkyl, C₁₋₄ alkoxy, F, Cl, Br, and OH;
R is H, C₁₋₈ alkyl, or aryl-C₁₋₄ alkyl, wherein the C₁₋₈ alkyl and aryl-C₁₋₄ alkyl are optionally substituted by 1, 2, or 3 substituents independently selected from C₁₋₄ alkyl, C₁₋₄ haloalkyl, halo, CN, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
R¹, R², R³, R⁴, and R⁵ are each independently selected from H, C₁₋₄ alkyl, C₁₋₄haloalkyl, C₂₋₆ alkenyl, C₂₋₆ alkynyl, C₃₋₄ cycloalkyl, 3-4 membered heterocycloalkyl, halo, CN, NO₂, N₃, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein the C₁₋₄ alkyl group of R¹, R², R³, R⁴, and R⁵ is optionally substituted by 1, 2, or 3 substituents independently selected from C₃₋₄ cycloalkyl, 3-4 membered heterocycloalkyl, halo, CN, NO₂, N₃, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
or R¹ and R² together with the carbon atoms to which they are attached form a fused C₃₋₇ cycloalkyl ring, a fused 3-7 membered heterocycloalkyl ring, a fused phenyl ring, or a fused 5-6 membered heteroaryl ring, each optionally substituted by 1, 2, or 3 substituents independently selected from C₁₋₄ alkyl, C₁₋₄haloalkyl, C₂₋₆ alkenyl, C₂₋₆ alkynyl, C₃₋₄ cycloalkyl, 3-4 membered heterocycloalkyl, halo, CN, NO₂, N₃, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR_{c1}R^{d1}, NR^{c1}C(O)R^{b1}, NR^{c1}C(O)OR^{a1}NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
or R² and R³ together with the carbon atoms to which they are attached form a fused C₃₋₇ cycloalkyl ring, a fused 3-7 membered heterocycloalkyl ring, a fused phenyl ring, or a fused 5-6 membered heteroaryl ring, each optionally substituted by 1, 2, or 3 substituents independently selected from C₁₋₄ alkyl, C₁₋₄haloalkyl, C₂₋₆ alkenyl, C₂₋₆ alkynyl, C₃₋₄ cycloalkyl, 3-4 membered heterocycloalkyl, halo, CN, NO₂, N₃, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR_{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
R⁶ and R⁷ are each independently selected from H, methyl, ethyl, CF₃, and CN;
each R^{A} is independently selected from halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}, wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄alkyl of R^{A} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2} NR^{c2}C(O)NR^{c2}R^{d2}, C(═NR^{e2})R^{b2}, C(═NR^{e2})NR^{c2}R^{d2}, NR^{c2}C(═R^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each R^{a}, R^{b}, R^{c}, R^{d}, R^{a1}, R^{b1}, R^{c1}, R^{d1}, R^{a2}, R^{b2}, R^{c2}, R^{d2}, R^{a3}, R^{b3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a}, R^{b}, R^{c}, R^{d}, R^{a1}, R^{b1}, R^{c1}, R^{d1}, R^{a2}, R^{b2}, R^{c2}, R^{d2}, R^{a3}, R^{b3}, R^{c3}, and R^{d3} is is optionally substituted with 1, 2, 3, 4, or 5 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
or R^{c} and R^{d} together with the N atom to which they are attached form a 4-7 membered heterocycloalkyl group optionally substituted with 1, 2, or 3 substituents independently selected from halo, C₁₋₄ alkyl, C₁₋₄haloalkyl, CN, OR^{a4}, SR^{a4}, C(O)R^{b4}, C(O)NR^{c4}R^{d4}, C(O)OR^{a4}OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
or R^{c1} and R^{d1} together with the N atom to which they are attached form a 4-7 membered heterocycloalkyl group optionally substituted with 1, 2, or 3 substituents independently selected from halo, C₁₋₄ alkyl, C₁₋₄haloalkyl, CN, OR^{a4}, SR^{a4}, C(O)R^{b4}, C(O)NR^{c4}R^{d4}, C(O)OR^{a4}OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
or R^{c2} and R^{d2} together with the N atom to which they are attached form a 4-7 membered heterocycloalkyl group optionally substituted with 1, 2, or 3 substituents independently selected from halo, C₁₋₄ alkyl, C₁₋₄haloalkyl, CN, OR^{a4}, SR^{a4}, C(O)R^{b4}, C(O)NR^{c4}R^{d4}, C(O)OR^{a4}OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
or R^{c3} and R^{d3} together with the N atom to which they are attached form a 4-7 membered heterocycloalkyl group optionally substituted with 1, 2, or 3 substituents independently selected from halo, C₁₋₄ alkyl, C₁₋₄haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
each R^{a4}, R^{b4}, R^{c4}, and R^{d4} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy; and
each R^{e}, R^{e1}, R^{e2}, R^{e3}, and R^{e4} is independently selected from H, C₁₋₄ alkyl, and CN;
wherein when A is phenyl optionally substituted with 1, 2, or 3 R^{A}, and L¹ is absent or C₁₋₂ alkylene, then L² is other than methylene optionally substituted by 1, 2, or 3 substituents independently selected from F, Cl, Br, OH, and methoxy.

2. (canceled)

3. The compound of claim 1, or a pharmaceutically acceptable salt thereof, wherein A is C₃₋₇ alkyl optionally substituted with 1 or 2 R^{A}.

4-5. (canceled)

6. The compound of claim 1, or a pharmaceutically acceptable salt thereof, wherein A is C₃₋₈ cycloalkyl optionally substituted with 1 R^{A}.

7-8. (canceled)

9. The compound of claim 1, or a pharmaceutically acceptable salt thereof, wherein A is 6 or 7-membered heterocycloalkyl optionally substituted with 1 R^{A}.

10-11. (canceled)

12. The compound of claim 1, or a pharmaceutically acceptable salt thereof, wherein A is phenyl optionally substituted with 1 R^{A}.

13. The compound of claim 1, or pharmaceutically acceptable salt thereof, wherein L¹ is absent.

14. The compound of claim 1, or pharmaceutically acceptable salt thereof, wherein L¹ is methylene.

15. The compound of claim 1, or pharmaceutically acceptable salt thereof, wherein L¹ is C₂₋₈ alkenylene, optionally substituted by 1 or 2 substituents independently selected from C₁₋₄ alkyl, C₁₋₄ haloalkyl, C₁₋₄ alkoxy, F, Cl, Br, and OH.

16. (canceled)

17. The compound of claim 1, or a pharmaceutically acceptable salt thereof, wherein L² is methylene optionally substituted by 1 or 2 substituents independently selected from C₁₋₄ alkyl, C₁₋₄ haloalkyl, C₁₋₄ alkoxy, F, Cl, Br, and OH.

18. (canceled)

19. The compound of claim 1, or a pharmaceutically acceptable salt thereof, wherein L² is C₃₋₈ cycloalkylene optionally substituted by 1, 2, or 3 substituents independently selected from C₁₋₄ alkyl, C₁₋₄haloalkyl, C₁₋₄ alkoxy, F, Cl, Br, and OH.

20-21. (canceled)

22. The compound of claim 1, or a pharmaceutically acceptable salt thereof, wherein R is H.

23-24. (canceled)

25. The compound of claim 1, or a pharmaceutically acceptable salt thereof, wherein R³ is C₁₋₄ haloalkyl.

26. (canceled)

27. The compound of claim 1, or a pharmaceutically acceptable salt thereof, wherein R¹, R², R⁴, and R⁵ are each H.

28. The compound of claim 1, or a pharmaceutically acceptable salt thereof, wherein R⁶ and R⁷ are each H.

29-30. (canceled)

31. The compound of claim 1, or a pharmaceutically acceptable salt thereof, wherein:
L¹ is absent, C₁₋₈ alkylene, or C₂₋₈ alkenylene, wherein the C₁₋₈ alkylene or C₂₋₈ alkenylene is optionally substituted by 1, 2, or 3 substituents independently selected from C₁₋₄ alkyl, C₁₋₄haloalkyl, C₁₋₄ alkoxy, F, Cl, Br, and OH;
L² is methylene or C₃₋₈ cycloalkylene, each optionally substituted by 1, 2, or 3 substituents independently selected from C₁₋₄ alkyl, C₁₋₄haloalkyl, C₁₋₄ alkoxy, F, Cl, Br, and OH;
R is H;
R¹, R², R⁴, and R⁵ are each H;
R³ is selected from C₁₋₄ alkyl, C₁₋₄haloalkyl, C₂₋₆ alkenyl, C₂₋₆ alkynyl, C₃₋₄ cycloalkyl, 3-4 membered heterocycloalkyl, halo, CN, NO₂, N₃, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein the C₁₋₄ alkyl group of R¹, R², R³, R⁴, and R⁵ is optionally substituted by 1, 2, or 3 substituents independently selected from C₃₋₄ cycloalkyl, 3-4 membered heterocycloalkyl, halo, CN, NO₂, N₃, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR_{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; and
R⁶ and R⁷ are each H.

32. (canceled)

33. The compound of claim 1, or a pharmaceutically acceptable salt thereof, wherein:
A is C₃₋₁₀ alkyl, C₃₋₁₀ cycloalkyl, 3-14 membered heterocycloalkyl, or phenyl, each optionally substituted with 1 R^{A};
L¹ is absent, methylene, or C₂₋₈ alkenylene, wherein the methylene or C₂₋₈ alkenylene is optionally substituted by 1 or 2 substituents independently selected from C₁₋₄ alkyl, C₁₋₄ alkoxy, and OH;
L² is methylene or C₃₋₈ cycloalkylene;
R is H;
R¹, R², R⁴, and R⁵ are each H;
R³ is C₁₋₄ haloalkyl;
R⁶ and R⁷ are each H;
each R^{A} is independently selected from C₁₋₆ alkyl, C₁₋₆ haloalkyl, and OR^{a2}; and
each R^{a2} is selected from H and C₁₋₆ alkyl.

34-39. (canceled)

40. The compound of claim 1 selected from:
2-(5-cyclooctyl-1-(4-(trifluoromethyl)benzyl)piperidin-3-yl)acetic acid;
2-(1-(4-(trifluoromethyl)benzyl)-5-(4-(trifluoromethyl)phenyl)piperidin-3-yl)cyclopropane-1-carboxylic acid;
2-(5-cycloheptyl-1-(4-(trifluoromethyl)benzyl)piperidin-3-yl)acetic acid;
2-(1-(4-(trifluoromethyl)benzyl)-5-(4-(trifluoromethyl)styryl)piperidin-3-yl)acetic acid;
2-(5-cyclohexyl-1-(4-(trifluoromethyl)benzyl)piperidin-3-yl)acetic acid; and
2-(5-isopropyl-1-(4-(trifluoromethyl)benzyl)piperidin-3-yl)acetic acid;
or a pharmaceuticlly acceptable salt thereof.

41. The compound of claim 1 selected from:
2-((3R,5S)-5-cyclooctyl-1-(4-(trifluoromethyl)benzyl)piperidin-3-yl)acetic acid;
2-((3S,5R)-5-cyclooctyl-1-(4-(trifluoromethyl)benzyl)piperidin-3-yl)acetic acid;
2-((3S,5S)-5-cyclooctyl-1-(4-(trifluoromethyl)benzyl)piperidin-3-yl)acetic acid;
2-((3R,5R)-5-cyclooctyl-1-(4-(trifluoromethyl)benzyl)piperidin-3-yl)acetic acid;
(2R)-2-((3S,5S)-1-(4-(trifluoromethyl)benzyl)-5-(4-(trifluoromethyl)phenyl)piperidin-3-yl)cyclopropane-1-carboxylic acid;
(2R)-2-((3R,5R)-1-(4-(trifluoromethyl)benzyl)-5-(4-(trifluoromethyl)phenyl)piperidin-3-yl)cyclopropane-1-carboxylic acid;
(2R)-2-((3S,5R)-1-(4-(trifluoromethyl)benzyl)-5-(4-(trifluoromethyl)phenyl)piperidin-3-yl)cyclopropane-1-carboxylic acid;
(2R)-2-((3R,5S)-1-(4-(trifluoromethyl)benzyl)-5-(4-(trifluoromethyl)phenyl)piperidin-3-yl)cyclopropane-1-carboxylic acid;
(2S)-2-((3S,5S)-1-(4-(trifluoromethyl)benzyl)-5-(4-(trifluoromethyl)phenyl)piperidin-3-yl)cyclopropane-1-carboxylic acid;
(2S)-2-((3R,5R)-1-(4-(trifluoromethyl)benzyl)-5-(4-(trifluoromethyl)phenyl)piperidin-3-yl)cyclopropane-1-carboxylic acid;
(2S)-2-((3S,5R)-1-(4-(trifluoromethyl)benzyl)-5-(4-(trifluoromethyl)phenyl)piperidin-3-yl)cyclopropane-1-carboxylic acid;
(2S)-2-((3R,5S)-1-(4-(trifluoromethyl)benzyl)-5-(4-(trifluoromethyl)phenyl)piperidin-3-yl)cyclopropane-1-carboxylic acid;
2-((3 S,5S)-1-(4-(trifluoromethyl)benzyl)-5-((E)-4-(trifluoromethyl)styryl)piperidin-3-yl)acetic acid;
2-((3R,5R)-1-(4-(trifluoromethyl)benzyl)-5-((E)-4-(trifluoromethyl)styryl)piperidin-3-yl)acetic acid;
2-((3S,5R)-1-(4-(trifluoromethyl)benzyl)-5-((E)-4-(trifluoromethyl)styryl)piperidin-3-yl)acetic acid;
2-((3R,5S)-1-(4-(trifluoromethyl)benzyl)-5-((E)-4-(trifluoromethyl)styryl)piperidin-3-yl)acetic acid;
2-((3R,5R)-5-cycloheptyl-1-(4-(trifluoromethyl)benzyl)piperidin-3-yl)acetic acid;
2-((3S,5S)-5-cycloheptyl-1-(4-(trifluoromethyl)benzyl)piperidin-3-yl)acetic acid;
2-((3R,5S)-5-cycloheptyl-1-(4-(trifluoromethyl)benzyl)piperidin-3-yl)acetic acid;
2-((3S,5R)-5-cycloheptyl-1-(4-(trifluoromethyl)benzyl)piperidin-3-yl)acetic acid;
2-((3R,5S)-5-cyclohexyl-1-(4-(trifluoromethyl)benzyl)piperidin-3-yl)acetic acid;
2-((3S,5R)-5-cyclohexyl-1-(4-(trifluoromethyl)benzyl)piperidin-3-yl)acetic acid;
2-((3R,5R)-5-cyclohexyl-1-(4-(trifluoromethyl)benzyl)piperidin-3-yl)acetic acid;
2-((3S,5S)-5-cyclohexyl-1-(4-(trifluoromethyl)benzyl)piperidin-3-yl)acetic acid;
2-((3R,5S)-5-isopropyl-1-(4-(trifluoromethyl)benzyl)piperidin-3-yl)acetic acid;
2-((3S,5R)-5-isopropyl-1-(4-(trifluoromethyl)benzyl)piperidin-3-yl)acetic acid;
2-((3R,5R)-5-isopropyl-1-(4-(trifluoromethyl)benzyl)piperidin-3-yl)acetic acid;
2-((3S,5S)-5-isopropyl-1-(4-(trifluoromethyl)benzyl)piperidin-3-yl)acetic acid;
or a pharmaceuticlly acceptable salt thereof.

42. A pharmaceutical composition comprising a compound of claim 1, and at least one pharmaceutically acceptable excipient.

43. A method of inhibiting an activity of XPO1 comprising contacting the XPO1 with a compound of claim 1, or a pharmaceutically acceptable salt thereof.

44. A method of treating or preventing a disease in a patient wherein the disease is characterized by overexpression or upregulation of XPO1 comprising administering to the patient a therapeutically effective amount of a compound of claim 1, or a pharmaceutically acceptable salt thereof.

45-48. (canceled)

49. A method of treating a solid tumor comprising administering to the patient a therapeutically effective amount of a compound of claim 1, or a pharmaceutically acceptable salt thereof.

50-51. (canceled)
